(19) United States
(12) Patent Application Publication
(10) Pub. No.: US 2013/0261022 A1
(43) Pub. Date: Oct. 3, 2013

(54) METABOLOMIC PROFILING OF PROSTATE CANCER

(71) Applicants: Arul M. Chinnaiyan, Plymouth, MI (US); Thekkelnaycke Rajendiran, Ann Arbor, MI (US)

(72) Inventors: Arul M. Chinnaiyan, Plymouth, MI (US); Thekkelnaycke Rajendiran, Ann Arbor, MI (US)

(73) Assignee: The Regents of The University of Michigan, Ann Arbor, MI (US)

(21) Appl. No.: 13/796,988

(22) Filed: Mar. 12, 2013

Related U.S. Application Data
(62) Division of application No. 12/976,177, filed on Dec. 22, 2010, now abandoned.
(60) Provisional application No. 61/289,206, filed on Dec. 22, 2009.

(51) Int. Cl. G01N 33/493

(52) U.S. Cl. CPC G01N 33/493
USPC 506/12

(57) ABSTRACT

The present invention relates to cancer markers. In particular, the present invention provides metabolites and panels of metabolites that are differentially present in cancer (e.g., prostate or breast cancer).

This application claims priority to application 61/289,206, filed Dec. 22, 2009.

### STATEMENT REGARDING FEDERALLY SPONSORED RESEARCH OR DEVELOPMENT

This invention was made with government support under Grant number U01 CA111275 from the National Institutes of Health. The government has certain rights in the invention.

### FIELD OF THE INVENTION

The present invention relates to cancer markers. In particular, the present invention provides metabolites and panels of metabolites that are differentially present in cancer (e.g., prostate or breast cancer).

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]; Maattanen et al., Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional serum and tissue biomarkers to supplement PSA screening is needed.

### SUMMARY OF THE INVENTION

The present invention relates to cancer markers. In particular, the present invention provides metabolites and panels of metabolites that are differentially present in cancer (e.g., prostate or breast cancer).

For example, in some embodiments, the present invention provides a method of diagnosing prostate or breast cancer, comprising: detecting the presence or absence of one or more (e.g., 2 or more, 3 or more, 5 or more, 10 or more, etc. measured together in a multiplex or panel format) cancer specific metabolites (e.g., pipecolic acid or fatty acids (including but not limited to myristic acid, palmitic acid, arachidonic acid, stearic acid, lauric acid, oleic acid) or polyamines (e.g., putrescine, spermidine, spermine)) in a sample (e.g., a tissue (e.g., biopsy) sample, a blood sample, a serum sample, or a urine sample) from a subject; and diagnosing the prostate or breast cancer based on the presence or absence of the cancer specific metabolite. In some embodiments, the cancer specific metabolite is present in cancerous samples but not non-cancerous samples. In some embodiments, the cancer specific metabolite is absent in cancerous samples but not non-cancerous samples. In some embodiments, one or more additional cancer markers are detected (e.g., in a panel or multiplex format) along with the cancer specific metabolites.

In some embodiments, the present invention provides a method of diagnosing prostate cancer, comprising: detecting the level of sarcosine, glutamic acid, glycine and cysteine in a urine sample from a subject; and diagnosing prostate cancer when the levels of sarcosine, glutamic acid, glycine and cysteine are elevated relative to the level in a non-cancerous subject. In some embodiments, the method further comprises the step of detecting the level of one or more metabolites selected from, for example, acetyl glucosamine, kyurenine, uracil, homocysteine, asparagine, glutamic acid, sperminide, spermine, 2-aminoadipic acid, leucine, proline, threonine, maleate, histidine, citrulline, adenosine and inosine.

The present invention further provides a method of characterizing prostate or breast cancer, comprising: detecting the presence or absence of an elevated level of a cancer-specific metabolite (e.g., pipecolic acid or fatty acids (including but not limited to myristic acid, palmitic acid, arachidonic acid, stearic acid, lauric acid, oleic acid) or polyamines (e.g., putrescine, spermidine, spermine)) in a sample (e.g., a tissue sample, a blood sample, a serum sample, a urine sample, a urine sediment sample) from a subject diagnosed with cancer; and characterizing the prostate or breast cancer based on the presence or absence of a cancer-specific metabolite. In some embodiments, the presence of an elevated level of fatty acid (e.g., to myristic acid, palmitic acid, arachidonic acid, stearic acid, lauric acid, oleic acid) in the sample is indicative of invasive prostate cancer in the subject. In some embodiments, the presence of an elevated level of pipecolic acid in the sample is indicative of invasive prostate cancer in the subject. In some embodiments, the presence of a reduced level of one or more polyamines (e.g., putrescine, spermidine, spermine) in a prostate tissue sample (e.g., prostate biopsy sample) is indicative of prostate cancer In some embodiments, the presence of an increased level of one or more polyamines (e.g, putrescine, spermidine, spermine) in a urine sample is indicative of prostate cancer.

In certain embodiments, the present invention provides a method of diagnosing breast cancer, comprising: detecting the presence or absence of one or more cancer specific metabolites such as pipecolic acid, serine, a polyamine, and a fatty acid in a sample from a subject; and diagnosing breast cancer based on the presence or absence of the cancer specific metabolite. In some embodiments, the polyamine is a polyamine such as putrescine, spermidine, and spermine. In some embodiments, the fatty acid a type such as myristic acid, palmitic acid, arachidonic acid, stearic acid, lauric acid, and oleic acid. In some embodiments, the sample is a type such as a tissue sample, a blood sample, a serum sample, and a urine sample. In some embodiments, the tissue sample is a biopsy sample. In some embodiments, the one or more cancer specific metabolites are present in cancerous samples but not non-cancerous samples. In some embodiment, the one or more cancer specific metabolites are absent in cancerous samples but present in non-cancerous samples. In some embodiments, the method comprises detection of the presence or absence of more than one said cancer specific metabolites simultaneously.

In certain embodiments, the present invention provides a method of characterizing breast cancer, comprising: detecting the presence or absence of one or more cancer specific metabolites such as pipecolic acid, serine, a polyamine, and a fatty acid in a sample from a subject; and characterizing the breast cancer based on the presence or absence of the cancer specific metabolite. In some embodiments, the polyamine is a polyamine such as putrescine, spermidine, and spermine. In some embodiments, the fatty acid is a fatty acid such as myristic acid, palmitic acid, arachidonic acid, stearic acid, lauric acid, and oleic acid. In some embodiments, the sample is a type such as tissue sample, a blood sample, a serum sample, and a urine sample. In some embodiments, the tissue sample is a biopsy sample. In some embodiments, the presence of an elevated level of the one or more cancer specific metabolites in the sample is indicative of breast cancer in the subject. In some embodiments, the presence of a lowered level of the one or more cancer specific metabolites in the sample is indicative of breast cancer in the subject. In some embodiments, method comprises detection of the presence or absence of more than one cancer specific metabolites simultaneously.

In certain embodiments, the present invention provides a method of diagnosing prostate cancer, comprising: detecting the presence or absence of one or more cancer specific metabolites such as pipecolic acid, serine, a polyamine, and a fatty acid in a urine sample from a subject; and diagnosing prostate cancer based on the presence or absence of the cancer specific metabolite in the urine sample. In some embodiments, the polyamine is a polyamine such as putrescine, spermidine, and spermine. In some embodiments, the fatty acid a fatty acid such as myristic acid, palmitic acid, arachidonic acid, stearic acid, lauric acid, and oleic acid. In some embodiments, the urine sample a urine sediment sample. In some embodiments, the one or more cancer specific metabolites are present in cancerous samples but not non-cancerous samples. In some embodiments, the one or more cancer specific metabolites are absent in cancerous samples but present in non-cancerous samples. In some embodiments, the method comprises the detection of the presence or absence of more than one cancer specific metabolites simultaneously.

In certain embodiments, the present invention provides a method of diagnosing prostate cancer, comprising: detecting a decrease in the level of one or more polyamines in a prostate tissue sample; and diagnosing prostate cancer based on the decrease in the level of one or more polyamines in the prostate tissue sample. In some embodiments, the prostate tissue sample is a biopsy sample. In some embodiments, the polyamine is a type such as putrescine, spermidine, and spermine.

In certain embodiments, the present invention provides a method of diagnosing prostate cancer, comprising: detecting an increase in the level of one or more polyamines in a urine sample; and diagnosing prostate cancer based on the decrease in the level of one or more polyamines in the urine sample. In some embodiments, the urine sample is a urine sediment sample. In some embodiments, the polyamine is a type such as putrescine, spermidine, and spermine.

In further embodiments, the present invention provides compositions, kits and systems for use in detecting levels of metabolites. In some embodiments, kits and systems comprise components necessary, sufficient or useful in detecting level of metabolites.

Additional embodiments of the present invention are described in the detailed description and experimental sections below.

### DESCRIPTION OF THE FIGURES

FIG. 1 shows that levels of glutamic acid are elevated in localized cancer and metastatic prostate cancer tissue samples in comparison to benign prostate tissues.

FIG. 2 shows that levels of glycine are elevated in localized cancer and metastatic prostate cancer tissue samples in comparison to benign prostate tissues.

FIG. 3 shows that levels of cysteine are elevated in localized cancer and metastatic prostate cancer tissue samples in comparison to benign prostate tissues.

FIG. 4 shows that levels of thymine are elevated in metastatic prostate cancer tissue samples in comparison to

FIG. 5 shows that levels of pipecolic acid are elevated in metastatic prostate cancer tissue samples in comparison to benign prostate tissues.

FIG. 6 shows that levels of uracil are elevated in localized cancer and metastatic prostate cancer tissue samples in comparison to benign prostate tissues.

FIG. 7 shows that levels of serine do not vary among benign, localized cancer, and metastatic prostate cancer tissue samples.

FIG. 8 shows that pipecolic acid levels are elevated in invasive prostate cancer cell lines (VCAP, Du145, and 2RVV1) compared to a non-invasive prostate cell line (RWPE).

FIG. 9 shows that invasive prostate cancer cell lines (LnCaP, Du145, PC3, 2RVV1) possess higher levels of uracil compared to a non-invasive prostate cell line (RWPE).

FIG. 10 shows that urine sediment samples from prostate biopsy-positive patients show higher sarcosine levels than urine sediment samples from prostate biopsy-negative patients.

FIG. 11 shows that urine sediment samples from prostate biopsy-positive patients show higher glutamic acid levels than urine sediment samples from prostate biopsy-negative patients.

FIG. 12 shows that urine sediment samples from prostate biopsy-positive patients show higher glycine levels than urine sediment samples from prostate biopsy-negative patients.

FIG. 13 shows that urine sediment samples from prostate biopsy-positive patients show higher cysteine levels than urine sediment samples from prostate biopsy-negative patients.

FIG. 14 shows that urine sediment samples from prostate biopsy-positive patients show equivalent methionine levels than urine sediment samples from prostate biopsy-negative patients.

FIG. 15 shows box plots indicating elevated levels of glutamic acid, glycine, and cysteine in prostate biopsy positive urine sediment samples compared to prostate biopsy negative controls.

FIG. 16 shows that metastatic prostate tissue samples have lower levels of spermine as compared to benign and localized prostate cancer samples.

FIG. 17 shows that metastatic prostate tissue samples have lower levels of putrescine as compared to benign and localized prostate cancer samples.

FIG. 18 shows that metastatic prostate tissue samples have lower levels of spermidine as compared to benign and localized prostate cancer samples.

FIG. 19 shows box plots of spermine, putrescine, and spermidine levels in benign, localized cancer, and metastatic prostate cancer tissue samples.

FIG. 20 shows that a non-invasive prostate cell line (RWPE) shows higher levels of spermine, putrescine, and spermidine as compared to invasive prostate cancer cell lines (VCAP, LnCaP, DU145, PC3, and 2RVV1).

FIG. 21 shows that spermine/methionine ratios are higher in urine sediment samples from biopsy-positive prostate cancer patients as compared to urine sediment samples from biopsy-negative controls.

FIG. 22 shows that spermidine/methionine ratios are higher in urine sediment samples from biopsy-positive prostate cancer patients as compared to urine sediment samples from biopsy-negative controls.

FIG. 23 shows box plots of spermine/methionine and spermidine/methionine ratios in urine sediments samples from biopsy-positive prostate cancer patients and biopsy-negative controls.

FIG. 24 shows that levels of myristic acid are elevated in localized and metastatic prostate cancer tissue samples as compared to benign controls.

FIG. 25 shows that levels of palmitic acid are elevated in localized and metastatic prostate cancer tissue samples as compared to benign controls.

FIG. 26 shows that levels of arachidonic acid are elevated in localized and metastatic prostate cancer tissue samples as compared to benign controls.

FIG. 27 shows that levels of stearic acid are elevated in metastatic prostate cancer tissue samples as compared to benign controls.

FIG. 28 shows that levels of lauric acid are elevated in metastatic prostate cancer tissue samples as compared to benign controls.

FIG. 29 shows that levels of oleic acid are elevated in metastatic prostate cancer tissue samples as compared to benign controls.

FIG. 30 shows box plots indicating levels of palmitic acid, myristic acid, stearic acid, arachidonic acid, oleic acid, and lauric acid in benign, localized cancer, and metastatic prostate cancer tissue samples.

FIG. 31 shows elevated sarcosine levels in breast cancer tissue samples as compared to benign tissue samples.

FIG. 32 shows that invasive breast cancer cell lines (MDA-MB-231, BT-549, T578, SVM-245) have elevated levels of sarcosine as compared to a non-invasive cell line (HME).

FIG. 33 shows that invasive breast cancer cell lines (MCF7, MDA-MB-231, T470, SKBR3) have elevated levels of putrescine, spermidine, and spermine as compared to a non-invasive cell line (MCF10A).

FIG. 34 shows a box-plot showing the levels of metabolites (sarcosine, glutamic acid, glycine and cysteine) based on GC-MS analysis in 70 post DRE urine sediments (35 Bx−ve and 35 Bx+ve).

FIG. 35 shows the ROC curves for a multiplex panel developed using logistic regression on the training set of 70 urine sediments consisting of 4 metabolites (sarcosine, glutamic acid, glycine and cysteine).

FIG. 36 shows a box-plot showing the levels of metabolites based on GC-MS analysis in prostate cancer tissues.

FIG. 37 shows that prostate cancer tissues show higher levels of leucine.

FIG. 38 shows that prostate cancer tissues show higher levels of asparagine.

FIG. 39 shows that prostate cancer tissues show higher levels of tryptophan.

FIG. 40 shows that prostate cancer tissues show higher levels of kynurenine.

FIG. 41 shows that prostate cancer tissues show higher levels of 3-aminobutyric acid.

FIG. 42 shows that tiopsy positive urine sediments show elevated levels of sarcosine.

FIG. 43 shows that biopsy positive urine sediment show higher levels of uracil (uracil/ala ratio).

FIG. 44 shows sarcosine reproducibility (independent prep).

FIG. 45 shows sarcosine reproducibility (replicates).

FIG. 46 shows stability of sarcosine in post DRE urine sediments.

FIG. 47 shows reproducibility of glutamic acid, glycine and cysteine in two independent preps.

### DEFINITIONS

To facilitate an understanding of the present invention, a number of terms and phrases are defined below:

“Prostate cancer” refers to a disease in which cancer develops in the prostate, a gland in the male reproductive system. “Low grade” or “lower grade” prostate cancer refers to non-metastatic prostate cancer, including malignant tumors with low potential for metastasis (e.g., prostate cancer that is considered to be less aggressive). “High grade” or “higher grade” prostate cancer refers to prostate cancer that has metastasized in a subject, including malignant tumors with high potential for metastasis (prostate cancer that is considered to be aggressive).

As used herein, the term “cancer specific metabolite” refers to a metabolite that is differentially present in cancerous cells compared to non-cancerous cells. For example, in some embodiments, cancer specific metabolites are present in cancerous cells but not non-cancerous cells. In other embodiments, cancer specific metabolites are absent in cancerous cells but present in non-cancerous cells. In still further embodiments, cancer specific metabolites are present at different levels (e.g., higher or lower) in cancerous cells as compared to non-cancerous cells. For example, a cancer specific metabolite may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (e.g., absent). A cancer specific metabolite is preferably differentially present at a level that is statistically significant (e.g., a p-value less than 0.05 and/or a q-value of less than 0.10 as determined using either Welch's T-test or Wilcoxon's rank-sum Test). Exemplary cancer specific metabolites are described in the detailed description and experimental sections below.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. A biological sample may contain any biological material suitable for detecting the desired biomarkers, and may comprise cellular and/or non-cellular material from a subject. The sample can be isolated from any suitable biological tissue or fluid such as, for example, prostate tissue, blood, blood plasma, urine, or cerebral spinal fluid (CSF).

A “reference level” of a metabolite means a level of the metabolite that is indicative of a particular disease state, phenotype, or lack thereof, as well as combinations of disease states, phenotypes, or lack thereof. A “positive” reference level of a metabolite means a level that is indicative of a particular disease state or phenotype. A “negative” reference level of a metabolite means a level that is indicative of a lack of a particular disease state or phenotype. For example, a “prostate cancer-positive reference level” of a metabolite means a level of a metabolite that is indicative of a positive diagnosis of prostate cancer in a subject, and a “prostate cancer-negative reference level” of a metabolite means a level of a metabolite that is indicative of a negative diagnosis of prostate cancer in a subject. A “reference level” of a metabolite may be an absolute or relative amount or concentration of the metabolite, a presence or absence of the metabolite, a range of amount or concentration of the metabolite, a minimum and/or maximum amount or concentration of the metabolite, a mean amount or concentration of the metabolite, and/or a median amount or concentration of the metabolite; and, in addition, “reference levels” of combinations of metabolites may also be ratios of absolute or relative amounts or concentrations of two or more metabolites with respect to each other. Appropriate positive and negative reference levels of metabolites for a particular disease state, phenotype, or lack thereof may be determined by measuring levels of desired metabolites in one or more appropriate subjects, and such reference levels may be tailored to specific populations of subjects (e.g., a reference level may be age-matched so that comparisons may be made between metabolite levels in samples from subjects of a certain age and reference levels for a particular disease state, phenotype, or lack thereof in a certain age group). Such reference levels may also be tailored to specific techniques that are used to measure levels of metabolites in biological samples (e.g., LC-MS, GC-MS, etc.), where the levels of metabolites may differ based on the specific technique that is used.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

“Mass Spectrometry” (MS) is a technique for measuring and analyzing molecules that involves fragmenting a target molecule, then analyzing the fragments, based on their mass/charge ratios, to produce a mass spectrum that serves as a “molecular fingerprint”. Determining the mass/charge ratio of an object is done through means of determining the wavelengths at which electromagnetic energy is absorbed by that object. There are several commonly used methods to determine the mass to charge ration of an ion, some measuring the interaction of the ion trajectory with electromagnetic waves, others measuring the time an ion takes to travel a given distance, or a combination of both. The data from these fragment mass measurements can be searched against databases to obtain definitive identifications of target molecules. Mass spectrometry is also widely used in other areas of chemistry, like petrochemistry or pharmaceutical quality control, among many others.

The term “metabolism” refers to the chemical changes that occur within the tissues of an organism, including “anabolism” and “catabolism”. Anabolism refers to biosynthesis or the buildup of molecules and catabolism refers to the breakdown of molecules.

A “metabolite” is an intermediate or product resulting from metabolism. Metabolites are often referred to as “small molecules”.

The term “metabolomics” refers to the study of cellular metabolites.

A “biopolymer” is a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another.

As used herein, the term “post-surgical tissue” refers to tissue that has been removed from a subject during a surgical procedure. Examples include, but are not limited to, biopsy samples, excised organs, and excised portions of organs.

As used herein, the terms “detect”, “detecting”, or “detection” may describe either the general act of discovering or discerning or the specific observation of a detectably labeled composition.

As used herein, the term “clinical failure” refers to a negative outcome following prostatectomy. Examples of outcomes associated with clinical failure include, but are not limited to, an increase in PSA levels (e.g., an increase of at least 0.2 ng ml⁻¹) or recurrence of disease (e.g., metastatic prostate cancer) after prostatectomy.

As used herein, the term “multiplex” refers to the detection of more than one substance (e.g., analyte, metabolite, compound) in a sample simultaneously.

### DETAILED DESCRIPTION OF THE INVENTION

The present invention relates to cancer markers. In particular embodiments, the present invention provides metabolites that are differentially present in cancer (e.g., prostate or breast cancer). Experiments conducted during the course of development of embodiments of the present invention indentified, for example, sarcosine, cysteine, glutamate, asparagine, glycine, leucine, acetyl glucosamine, homocysteine, proline, threonine, histidine, n-acetyl-aspartic acid, maleate, citrulline, inosine, inositol, adenosine, taurine, creatine, uric acid, glutathione, uracil, kynurenine, glycerol-s-phosphate, glycocholic acid, suberic acid, thymine, glutamic acid, serine, uracil, xanthosisne, 4-acetamidobutyric acid, pipecolic acid, palmitic acid, stearic acid, lauric acid, oleic acid, aracidonic acid, methionine, spermine, tryptophan, 2-aminoadipic acid, 3-aminoisobutyric acid, spermadine, putrescine, myristic acid and thymine as differentially present in subjects with cancer. Additional markers are described herein and in WO 2009/026152 and WO 2008/036691, each of which is herein incorporated by reference in its entirety.

The disclosed markers find use as diagnostic, research, screening and therapeutic targets. In some embodiments, the present invention provides compositions and methods for diagnosing cancer (e.g., prostate or breast cancer). In some embodiments, the present invention provides methods of identifying invasive cancers (e.g., invasive prostate cancer, invasive breast cancer) based on the presence of elevated levels of metabolites.

Embodiments of the present invention provide panels (e.g., comprising 2 or more, 5 or more, 10 or more, 25 or more or 50 or more) markers useful in diagnostic, prognostic, screening or therapeutic applications.

### I. Diagnostic and Screening Applications

In some embodiments, the present invention provides methods and compositions for diagnosing and screening for cancer (e.g., prostate or breast cancer), including but not limited to, characterizing or diagnosing risk of cancer, presence or absence stage of cancer, risk of or presence of metastasis, invasiveness of cancer, etc. based on the presence of cancer specific metabolites or their derivates, precursors, metabolites, etc. Exemplary diagnostic methods are described below.

A. Sample

Any patient sample suspected of containing cancer-specific metabolites is tested according to the methods described herein. By way of non-limiting examples, the sample may be tissue (e.g., a prostate or breast biopsy sample or post-surgical tissue), blood, urine, or a fraction thereof (e.g., plasma, serum, urine supernatant, urine cell pellet, urine sediment, or prostate cells). In some embodiments, the sample is a tissue sample obtained from a biopsy or following surgery (e.g., prostate biopsy). A urine sample is preferably collected immediately following an attentive digital rectal examination (DRE), which causes prostate cells from the prostate gland to shed into the urinary tract.

In some embodiments, the patient sample undergoes preliminary processing designed to isolate or enrich the sample for cancer specific metabolites or cells that contain cancer specific metabolites. A variety of techniques known to those of ordinary skill in the art may be used for this purpose, including but not limited: centrifugation; immunocapture; and cell lysis.

B. Detection of Metabolites

Metabolites may be detected using any suitable method including, but not limited to, liquid and gas phase chromatography, alone or coupled to mass spectrometry (See e.g., experimental section below), NMR (See e.g., US patent publication 20070055456, herein incorporated by reference), immunoassays, chemical assays, spectroscopy and the like. In some embodiments, commercial systems for chromatography and NMR analysis are utilized.

In other embodiments, metabolites (e.g., biomarkers and derivatives thereof) are detected using optical imaging techniques such as magnetic resonance spectroscopy (MRS), magnetic resonance imaging (MRI), CAT scans, ultra sound, MS-based tissue imaging or X-ray detection methods (e.g., energy dispersive x-ray fluorescence detection).

Any suitable method may be used to analyze the biological sample in order to determine the presence, absence or level(s) of the one or more metabolites in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, biochemical or enzymatic reactions or assays, and combinations thereof. Further, the level(s) of the one or more metabolites may be measured indirectly, for example, by using an assay that measures the level of a compound (or compounds) that correlates with the level of the biomarker(s) that are desired to be measured.

The levels of one or more of the recited metabolites may be determined in the methods of the present invention. For example, the level(s) of one metabolites, two or more metabolites, three or more metabolites, four or more metabolites, five or more metabolites, six or more metabolites, seven or more metabolites, eight or more metabolites, nine or more metabolites, ten or more metabolites, etc., including a combination of some or all of the metabolites described herein may be determined and used in such methods. Determining levels of combinations of the metabolites may allow greater sensitivity and specificity in the methods, such as diagnosing prostate cancer and aiding in the diagnosis of prostate cancer, and may allow better differentiation or characterization of prostate cancer from other prostate disorders (e.g. benign prostatic hypertrophy (BPH), prostatitis, etc.) or other cancers that may have similar or overlapping metabolites to prostate cancer (as compared to a subject not having prostate cancer). For example, ratios of the levels of certain metabolites in biological samples may allow greater sensitivity and specificity in diagnosing prostate cancer and aiding in the diagnosis of prostate cancer and allow better differentiation or characterization of prostate cancer from other cancers or other disorders of the prostate that may have similar or overlapping metabolites to prostate cancer (as compared to a subject not having prostate cancer). In some embodiments, the level of one or more metabolites (e.g., pipecolic acid or fatty acids (including but not limited to myristic acid, palmitic acid, arachidonic acid, stearic acid, lauric acid, oleic acid)) finds use in the differentiation or characterization of breast cancer.

C. Data Analysis

In some embodiments, a computer-based analysis program is used to translate the raw data generated by the detection assay (e.g., the presence, absence, or amount of a cancer specific metabolite) into data of predictive value for a clinician. The clinician can access the predictive data using any suitable means. Thus, in some embodiments, the present invention provides the further benefit that the clinician, who is not likely to be trained in metabolite analysis, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a blood, urine or serum sample) is obtained from a subject and submitted to a profiling service (e.g., clinical lab at a medical facility, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., metabolic profile), specific for the diagnostic or prognostic information desired for the subject.

The profile data is then prepared in a format suitable for interpretation by a treating clinician. For example, rather than providing raw data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

In some embodiments, elevated or decreased levels of metabolites (e.g., relative to the level in normal prostate cells, increase in level relative to a prior time point, increase relative to a pre-established threshold level, etc.) are indicative of cancer in the sample.

When the amount(s) or level(s) of the one or more metabolites in the sample are determined, the amount(s) or level(s) may be compared to cancer metabolite-reference levels, such as cancer-positive and/or cancer-negative reference levels to aid in diagnosing or to diagnose whether the subject has cancer. Levels of the one or more metabolites in a sample corresponding to the cancer-positive reference levels (e.g., levels that are the same as the reference levels, substantially the same as the reference levels, above and/or below the minimum and/or maximum of the reference levels, and/or within the range of the reference levels) are indicative of a diagnosis of cancer in the subject. Levels of the one or more metabolites in a sample corresponding to the cancer-negative reference levels (e.g., levels that are the same as the reference levels, substantially the same as the reference levels, above and/or below the minimum and/or maximum of the reference levels, and/or within the range of the reference levels) are indicative of a diagnosis of no cancer in the subject. In addition, levels of the one or more metabolites that are differentially present (especially at a level that is statistically significant) in the sample as compared to cancer-negative reference levels are indicative of a diagnosis of cancer in the subject. Levels of the one or more metabolites that are differentially present (especially at a level that is statistically significant) in the sample as compared to cancer-positive reference levels are indicative of a diagnosis of no cancer in the subject.

The level(s) of the one or more metabolites are compared to cancer-positive and/or prostate cancer-negative reference levels using various techniques, including a simple comparison (e.g., a manual comparison) of the level(s) of the one or more metabolites in the biological sample to cancer-positive and/or cancer-negative reference levels. The level(s) of the one or more metabolites in the biological sample may also be compared to cancer-positive and/or cancer-negative reference levels using one or more statistical analyses (e.g., t-test, Welch's T-test, Wilcoxon's rank sum test, random forest).

D. Compositions & Kits

Compositions for use (e.g., sufficient for, necessary for, or useful for) in the diagnostic, research or screening methods of some embodiments of the present invention include reagents necessary, sufficient or useful for detecting the presence or absence of cancer specific metabolites. Any of these compositions, alone or in combination with other compositions of the present invention, may be provided in the form of a kit. Kits may further comprise appropriate controls and/or detection reagents.

E. Panels

Embodiments of the present invention provide for multiplex or panel assays that simultaneously detect one or more of the markers of the present invention (e.g., sarcosine, cysteine, glutamate, asparagine, glycine, leucine, proline, threonine, histidine, n-acetyl-aspartic acid, inosine, inositol, adenosine, taurine, creatine, uric acid, glutathione, uracil, kynurenine, glycerol-s-phosphate, glycocholic acid, suberic acid, thymine, glutamic acid, xanthosine, 4-acetamidobutyric acid, n-acetyltyrosine and thymine, pipecolic acid, fatty acids (including but not limited to myristic acid, palmitic acid, arachidonic acid, stearic acid, lauric acid, oleic acid) or polyamines (including but not limited to putrescine, spermidine, spermine)), alone or in combination with additional cancer markers known in the art. For example, in some embodiments, panel or combination assays are provided that detected 2 or more, 3 or more, 4 or more, 5 or more, 6 or more, 7 or more, 8 or more, 9 or more, 10 or more, 15 or more, or 20 or more markers in a single assay. In some embodiments, assays are automated or high throughput.

In some embodiments, the panel comprises sarcosine, glutamic acid, glycine and cysteine. Experiments conducted during the course of development of embodiments of the present invention determined that a panel of sarcosine, glutamic acid, glycine and cysteine demonstrated a higher area under the curve that any individual marker and is thus more sensitive in detecting prostate cancer. In some embodiments, panels for detection of prostate cancer further comprise one or more of acetyl glucosamine, kyurenine, uracil, homocysteine, asparagine, glutamic acid, sperminide, spermine, 2-aminoadipic acid, leucine, proline, threonine, maleate, histidine, citrulline, adenosine or inosine.

In some embodiments, additional cancer markers are included in multiplex or panel assays. Markers are selected for their predictive value alone or in combination with the metabolic markers described herein. Exemplary prostate cancer markers include, but are not limited to: AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); and, those disclosed in U.S. Pat. Nos. 5,854,206 and 6,034,218, and U.S. Publication No. 20030175736, each of which is herein incorporated by reference in its entirety. Markers for other cancers, diseases, infections, and metabolic conditions are also contemplated for inclusion in a multiplex or panel format.

### II. Therapeutic Methods

In some embodiments, the present invention provides therapeutic methods (e.g., that target the cancer specific metabolites described herein). In some embodiments, the therapeutic methods target enzymes or pathway components of the cancer specific metabolites described herein.

For example, in some embodiments, the present invention provides compounds that target the cancer specific metabolites of the present invention. The compounds may decrease the level of cancer specific metabolite by, for example, interfering with synthesis of the cancer specific metabolite (e.g., by blocking transcription or translation of an enzyme involved in the synthesis of a metabolite, by inactivating an enzyme involved in the synthesis of a metabolite (e.g., by post translational modification or binding to an irreversible inhibitor), or by otherwise inhibiting the activity of an enzyme involved in the synthesis of a metabolite) or a precursor or metabolite thereof, by binding to and inhibiting the function of the cancer specific metabolite, by binding to the target of the cancer specific metabolite (e.g., competitive or non competitive inhibitor), or by increasing the rate of break down or clearance of the metabolite. The compounds may increase the level of cancer specific metabolite by, for example, inhibiting the break down or clearance of the cancer specific metabolite (e.g., by inhibiting an enzyme involved in the breakdown of the metabolite), by increasing the level of a precursor of the cancer specific metabolite, or by increasing the affinity of the metabolite for its target. Exemplary therapeutic targets include, but are not limited to, glycine-N-methyl transferase (GNMT) and sarcosine.

A. Metabolic Pathways

The metabolic pathways of exemplary cancer specific metabolites are described below. Additional metabolites are contemplated for use in the compositions and methods of the present invention and are described, for example, in the Experimental section below.

i. Sarcosine Metabolism

For example, sarcosine is involved in choline metabolism in the liver. The oxidative degradation of choline to glycine in the mammalian liver takes place in the mitochondria, where it enters by a specific transporter. The two last steps in this metabolic pathway are catalyzed by dimethylglycine dehydrogenase (Me2GlyDH), which converts dimethylglycine into sarcosine, and sarcosine dehydrogenase (SarDH), which converts sarcosine (N-methylglycine) into glycine. Both enzymes are located in the mitochondrial matrix. Accordingly, in some embodiments, therapeutic compositions target Me2GlyDH and/or SarDH. Exemplary compounds are identified, for example, by using the drug screening methods described herein.

ii. Glycholic Acid Metabolism

The end products of cholesterol utilization are the bile acids, synthesized in the liver. Synthesis of bile acids is the predominant mechanisms for the excretion of excess cholesterol. However, the excretion of cholesterol in the form of bile acids is insufficient to compensate for an excess dietary intake of cholesterol. The most abundant bile acids in human bile are chenodeoxycholic acid (45%) and cholic acid (31%). The carboxyl group of bile acids is conjugated via an amide bond to either glycine or taurine before their secretion into the bile canaliculi. These conjugation reactions yield glycocholic acid and taurocholic acid, respectively. The bile canaliculi join with the bile ductules, which then form the bile ducts. Bile acids are carried from the liver through these ducts to the gallbladder, where they are stored for future use. The ultimate fate of bile acids is secretion into the intestine, where they aid in the emulsification of dietary lipids. In the gut the glycine and taurine residues are removed and the bile acids are either excreted (only a small percentage) or reabsorbed by the gut and returned to the liver. This process is termed the enterohepatic circulation.

iii. Suberic Acid Metabolism

Suberic acid, also octanedioic acid, is a dicarboxylic acid, with formula C₆H₁₂(COOH)₂. The peroxisomal metabolism of dicarboxylic acids results in the production of the mediumchain dicarboxylic acids adipic acid, suberic acid, and sebacic acid, which are excreted in the urine.

iv. Xanthosine Metabolism

Xanthosine is involved in purine nucleoside metabolism. Specifically, xanthosine is an intermediate in the conversion of inosine to guanosine. Xanthylic acid can be used in quantitative measurements of the Inosine monophosphate dehydrogenase enzyme activities in purine metabolism, as recommended to ensure optimal thiopurine therapy for children with acute lymphoblastic leukaemia (ALL).

v. Polyamine Metabolism

Polyamines have two or more primary amino groups, and are essential molecules in eukaryotes and prokaryotes. Though it is known that polyamines are synthesized in cells via highly-regulated pathways, their actual function is not entirely clear. As cations, they bind to DNA at regularly-spaced intervals.

If cellular polyamine synthesis is inhibited, cell growth is halted or severely retarded. Provision of exogenous polyamines restores the growth of these cells. Most eukaryotic cells have a polyamine transporter system on their cell membrane that facilitates the internalization of exogenous polyamines. This system is highly active in rapidly proliferating cells and is the target of some chemotherapeutics (e.g., DMFO, MGBG, BCNU, and analogs thereof).

Polyamines are also important modulators of a variety of ion channels, including NMDA receptors and AMPA receptors. They block inward-rectifier potassium channels, thereby conserving cellular energy, (K⁺ ion gradient across the cell membrane).

Examples of polyamines include but are not limited to putrescine, cadaverine, spermine, and spermidine. Putrescine is synthesized biologically via two different pathways, both starting from arginine. In one pathway, arginine is converted into agmatine, with a reaction catalyzed by the enzyme arginine decarboxylase (ADC); then agmatine is transformed into carbamilputrescine by agmatine imino hydroxylase (AIH). Finally, carbamilputrescine is converted into putrescine. In the second pathway, arginine is converted into ornithine and then ornithine is converted into putrescine by ornithine decarboxylase (ODC). Cadaverine is synthesized from lysine in a one-step reaction with lysine decarboxylase (LDC). Spermidine is synthesized from putrescine, using an aminopropylic group from decarboxylated S-adenosyl-L-methionine (SAM). The reaction is catalyzed by spermidine synthase. Spermine is synthesized from the reaction of spermidine with SAM in the presence of the enzyme spermine synthase.

vi. Fatty Acid Metabolism

Fatty acids comprise a carboxylic acid often with a long unbranched aliphatic tail (chain), which is either saturated or unsaturated. Examples of fatty acids include but are not limited to myristic acid, palmitic acid, arachidonic acid, stearic acid, lauric acid (also known as dodecanoic acid) and oleic acid.

Myristic acid, also called tetradecanoic acid or 14:0 is a common saturated fatty acid with the molecular formula CH₃(CH₂)₁₂COOH. A myristate is a salt or ester of myristic acid. Myristic acid is named after nutmeg (Myristica fragrans). Nutmeg butter is 75% trimyristin, the triglyceride of myristic acid. Besides nutmeg, myristic acid is also found in palm oil, coconut oil, butter fat, and spermacetin, the crystallized fraction of oil from the sperm whale. Myristic acid is also commonly added co-translationally to the penultimate, nitrogen-terminus, glycine in receptor-associated kinases to confer the membrane localisation of the enzyme. Myristic acid has a sufficiently high hydrophobicity to become incorporated into the fatty acyl core of the phospholipid bilayer of the plasma membrane of the eukaryotic cell. In this way, myristic acid acts as a lipid anchor in biomembranes.

Palmitic acid, CH₃(CH₂)₁₄COOH or hexadecanoic acid in IUPAC nomenclature, is one of the most common saturated fatty acids found in animals and plants, and is a major component of the oil from palm trees (palm oil and palm kernel oil). The word palmitic is from the French “palmitique”, the pith of the palm tree. Palmitic acid is the first fatty acid produced during lipogenesis (fatty acid synthesis) and from which longer fatty acids can be produced. Palmitate negatively feeds back on acetyl-CoA carboxylase (ACC) which is responsible for converting acetyl-CoA to malonyl-CoA which is used to add to the growing acyl chain, thus preventing further palmitate generation.

Arachidonic acid (also known as AA or ARA) is an omega-6 fatty acid 20:4(ω-6). It is the counterpart to the saturated arachidic acid found in peanut oil, (L. arachis—peanut). Arachidonic acid is a carboxylic acid with a 20-carbon chain and four cis double bonds; the first double bond is located at the sixth carbon from the omega end. ‘Arachidonic acid’ is occasionally used to designate any of the eicosatetraenoic acids. However, the term is commonly limited to all-cis 5,8,11,14-eicosatetraenoic acid. Arachidonic acid is freed from a phospholipid molecule by the enzyme phospholipase A2 (PLA₂), which cleaves off the fatty acid, but can also be generated from DAG by DAG lipase. Arachidonic acid generated for signaling purposes appears to be derived by the action of a phosphatidylcholine-specific cytosolic phospholipase A2 (cPLA₂, 85 kDa), whereas inflammatory arachidonic acid is generated by the action of a low-molecular-weight secretory PLA₂ (sPLA₂, 14-18 kDa). Arachidonic acid is a precursor in the production of eicosanoids: 1) the enzymes cyclooxygenase and peroxidase lead to Prostaglandin H2, which in turn is used to produce the prostaglandins, prostacyclin, and thromboxanes; 2) the enzyme 5-lipoxygenase leads to 5-HPETE, which in turn is used to produce the leukotrienes; 3) arachidonic acid is also used in the biosynthesis of anandamide; and 4) some arachidonic acid is converted into hydroxyeicosatetraenoic acids (HETEs) and epoxyeicosatrienoic acids (EETs) by epoxygenase. The production of these derivatives and their action in the body are collectively known as the arachidonic acid cascade.

Stearic acid or 18:0 is a saturated fatty acid. It is a waxy solid with chemical formula C₁₈H₃₆O₂, or CH₃(CH₂)₁₆COOH. Stearic acid undergoes the typical reactions of saturated carboxylic acids, notably reduction to stearyl alcohol, and esterification with a range of alcohols. Isotope labeling in humans (Emken et al. (1994) Am. J. Clin. Nutr. 60:1023 S-1328S) indicated that the fraction of dietary stearic acid oxidatively desaturated to oleic acid was 2.4 times higher than the fraction of palmitic acid analogously converted to palmitoleic acid. Also, stearic acid was less likely to be incorporated into cholesterol esters.

Oleic acid is a mono-unsaturated omega-9 fatty acid found in various animal and vegetable sources and is the most abundant fatty acid in human adipose tissue. It has the formula CH₃(CH₂)₇CH═CH(CH₂)₇COOH). The trans-isomer of oleic acid is called elaidic acid.

B. Small Molecule Therapies

In some embodiments, small molecule therapeutics are utilized. In certain embodiments, small molecule therapeutics targeting cancer specific metabolites. In some embodiments, small molecule therapeutics are identified, for example, using the drug screening methods of the present invention.

C. Nucleic Acid Based Therapies

In other embodiments, nucleic acid based therapeutics are utilized. Exemplary nucleic acid based therapeutics include, but are not limited to antisense RNA, siRNA, and miRNA. In some embodiments, nucleic acid based therapeutics target the expression of enzymes in the metabolic pathways of cancer specific metabolites (e.g., those described above).

In some embodiments, nucleic acid based therapeutics are antisense. siRNAs are used as gene-specific therapeutic agents (Tuschl and Borkhardt, Molecular Intervent. 2002; 2(3):158-67, herein incorporated by reference). The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

In other embodiments, expression of genes involved in metabolic pathways of cancer specific metabolites is modulated using antisense compounds that specifically hybridize with one or more nucleic acids encoding the enzymes (See e.g., Georg Sczakiel, Frontiers in Bioscience 5, d194-201 Jan. 1, 2000; Yuen et al., Frontiers in Bioscience d588-593, Jun. 1, 2000; Antisense Therapeutics, Second Edition, Phillips, M. Ian, Humana Press, 2004; each of which is herein incorporated by reference).

D. Gene Therapy

The present invention contemplates the use of any genetic manipulation for use in modulating the expression of enzymes involved in metabolic pathways of cancer specific metabolites described herein. Examples of genetic manipulation include, but are not limited to, gene knockout (e.g., removing the gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

E. Antibody Therapy

In some embodiments, the present invention provides antibodies that target cancer specific metabolites or enzymes involved in their metabolic pathways. Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, antibody based therapeutics are formulated as pharmaceutical compositions as described below. In preferred embodiments, administration of an antibody composition of the present invention results in a measurable decrease in cancer (e.g., decrease or elimination of tumor).

F. Pharmaceutical Compositions

The present invention further provides pharmaceutical compositions (e.g., comprising pharmaceutical agents that modulate the level or activity of cancer specific metabolites. The pharmaceutical compositions of some embodiments of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more nucleic acid compounds and (b) one or more other chemotherapeutic agents that function by different mechanisms. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the pharmaceutical composition is administered in maintenance doses, ranging from 0.01 μg to 100 g per kg of body weight, once or more daily, to once every 20 years.

### III. Drug Screening Applications

In some embodiments, the present invention provides drug screening assays (e.g., to screen for anticancer drugs). The screening methods of the present invention utilize cancer specific metabolites described herein. As described above, in some embodiments, test compounds are small molecules, nucleic acids, or antibodies. In some embodiments, test compounds target cancer specific metabolites directly. In other embodiments, they target enzymes involved in metabolic pathways of cancer specific metabolites.

In preferred embodiments, drug screening methods are high throughput drug screening methods. Methods for high throughput screening are well known in the art and include, but are not limited to, those described in U.S. 6468736, WO06009903, and U.S. 5972639, each of which is herein incorporated by reference.

The test compounds of some embodiments of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In some embodiments, the markers described herein are used to produce a model system for the identification of therapeutic agents for cancer. For example, a cancer-specific biomarker metabolite (for example, sarcosine which activates cell proliferation) can be added to a cell-line to increase the cancer aggressivity of the cell line. The cell line will have an improved dynamic range of response (e.g., ‘readout’) which is useful to screen for anti-cancer agents. While an in vitro example is described, the model assay system may be in vitro, in vivo or ex vivo.

### VII. Transgenic Animals

The present invention contemplates the generation of transgenic animals comprising an exogenous gene (e.g., resulting in altered levels of a cancer specific metabolite). In preferred embodiments, the transgenic animal displays an altered phenotype (e.g., increased or decreased presence of metabolites) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present invention and are not to be construed as limiting the scope thereof.

### Example 1

### Biomarkers Discovered in Urine

### I. General Methods

A. Identification of Metabolic Profiles for Prostate Cancer

Each sample was analyzed to determine the concentration of several hundred metabolites. Analytical techniques such as GC-MS (gas chromatography-mass spectrometry) and UHPLC-MS (ultra high performance liquid chromatography-mass spectrometry) were used to analyze the metabolites. Multiple aliquots were simultaneously, and in parallel, analyzed, and, after appropriate quality control (QC), the information derived from each analysis was recombined. Every sample was characterized according to several thousand characteristics, which ultimately amount to several hundred chemical species. The techniques used were able to identify novel and chemically unnamed compounds.

B. Statistical Analysis

The data was analyzed using T-tests to identify molecules (either known, named metabolites or unnamed metabolites) present at differential levels in a definable population or subpopulation (e.g., biomarkers for prostate cancer biological samples compared to control biological samples) useful for distinguishing between the definable populations (e.g., prostate cancer and control, low grade prostate cancer and high grade prostate cancer). Other molecules (either known, named metabolites or unnamed metabolites) in the definable population or subpopulation were also identified. In some analyses the data was normalized according to creatinine levels in the samples while in other analyses the samples were not normalized. Results of both analyses are included.

C. Biomarker identification

Various peaks identified in the analyses (e.g. GC-MS, UHPLC-MS, MS-MS), including those identified as statistically significant, were subjected to a mass spectrometry based chemical identification process. Biomarkers were discovered by (1) analyzing urine samples from different groups of human subjects to determine the levels of metabolites in the samples and then (2) statistically analyzing the results to determine those metabolites that were differentially present in the two groups.

Biomarkers that Distinguish Cancer from Non-Cancer:

The urine samples used for the analysis were from 51 control individuals with negative biopsies for prostate cancer, and 59 individuals with prostate cancer. After the levels of metabolites were determined, the data was analyzed using the Wilcoxon test to determine differences in the mean levels of metabolites between two populations (e.g., Prostate cancer vs. Control).

As listed below in Table 1, biomarkers were discovered that were differentially present between plasma samples from subjects with prostate cancer and Control subjects with negative prostate biopsies (e.g., not diagnosed with prostate cancer). Table 1 includes, for each listed biomarker, the p-value determined in the statistical analysis of the data concerning the biomarkers, the compound ID useful to track the compound in the chemical database and the analytical platform used to identify the compounds (GC refers to GC/MS and LC refers to UHPLC/MS/MS2). P-values that are listed as 0.000 are significant at p<0.0001. LCpos and LCneg refer to UHPLC separation using buffers and parameters that are optimized for detecting positive ions or negative ions, respectively.

TABLE 1
Biomarkers useful to distinguish cancer from non-cancer.

| | | | | % change |
|---|---|---|---|---|
| COMP_ID | COMPOUND | LIB_ID | p-value | in PCA |
| 34404 | 1,3-7-trimethyluric acid | LCneg | 0.0457 | −61.6700 |
| 32391 | 1,3-dimethylurate | GC | 0.0188 | 264.8018 |
| 34400 | 1-7-dimethylurate | LCneg | 0.0442 | −55.8508 |
| 15650 | 1-methyladenosine | LCpos | 0.0156 | 61.7971 |
| 31609 | 1-methylguanosine | LCpos | 0.0181 | 10.9223 |
| 34395 | 1-methylurate | LCpos | 0.047 | −30.4105 |
| 22030 | 2-hydroxyisobutyrate | GC | 0.0039 | 62.9593 |
| 1432 | 2-hydroxyphenylacetate | LCneg | 0.0344 | 59.6277 |
| 32776 | 2-methylbutyroylcarnitine- | LCpos | 0.0444 | 72.8112 |
| 1431 | 3-(4-hydroxyphenyl)lactate | GC | 0.003 | 33.8077 |
| 18296 | 3-4-dihydroxyphenylacetate | GC | 0.001 | 147.8039 |
| 1566 | 3-amino-isobutyrate | GC | 0.0167 | 272.4645 |
| 32654 | 3-dehydrocarnitine- | LCpos | 0.0188 | 56.2816 |
| 32397 | 3-hydroxy-2-ethylpropionate | GC | 0.0477 | 40.3754 |
| 531 | 3-hydroxy-3-methylglutarate | GC | 4.03E−05 | 37.8097 |
| 15673 | 3-hydroxybenzoate | LCneg | 3.00E−04 | 196.7772 |
| 12017 | 3-methoxytyrosine | LCpos | 0.0069 | 95.6504 |
| 31940 | 3-methylcrotonylglycine | LCpos | 0.0102 | 62.5089 |
| 1557 | 3-methylglutarate | GC | 0.0134 | 36.0177 |
| 15677 | 3-methylhistidine | LCneg | 0.0203 | −42.0713 |
| 3155 | 3-ureidopropionate | LCpos | 0.0056 | 68.9399 |
| 1558 | 4-acetamidobutanoate | LCpos | 0.0143 | 77.3732 |
| 22115 | 4-acetylphenyl-sulfate | LCneg | 0.0467 | 100.8052 |
| 21133 | 4-hydroxybenzoate | GC | 0.0049 | 62.6825 |
| 1568 | 4-hydroxymandelate | GC | 0.0091 | 120.1023 |
| 541 | 4-hydroxyphenylacetate | GC | 0.0036 | 85.2767 |
| 22118 | 4-ureidobutyrate | LCpos | 0.0134 | 67.8751 |
| 1418 | 5,6-dihydrothymine | GC | 0.0057 | 140.1535 |
| 1559 | 5,6-dihydrouracil | GC | 0.004 | 80.4881 |
| 437 | 5-hydroxyindoleacetate | GC | 1.00E−04 | 61.2357 |
| 1419 | 5-methylthioadenosine (MTA) | LCpos | 5.00E−04 | 20.5901 |
| 1494 | 5-oxoproline | LCpos | 0.0047 | 17.9299 |
| 31580 | 7-methylguanosine | GC | 1.00E−04 | 75.7087 |
| 554 | adenine | GC | 1.00E−04 | 46.4734 |
| 555 | adenosine | LCpos | 0.0011 | 30.8684 |
| 2831 | adenosine-3′,5′-cyclic-monophosphate | LCpos | 0.0038 | 75.5601 |
| | (cAMP) | | | |
| 1126 | alanineQUM | GC | 0.0419 | 66.0477 |
| 22808 | allantoin | GC | 0.0085 | 47.1337 |
| 15142 | allo-threonine | GC | 0.0148 | 198.5838 |
| 31591 | androsterone sulfate | LCneg | 0.016 | 96.0684 |
| 575 | arabinose | GC | 2.00E−04 | 67.9778 |
| 15964 | arabitol | GC | 7.00E−04 | 46.2583 |
| 1640 | ascorbate (Vitamin C) | GC | 0.0327 | 55.6234 |
| 18362 | azelate (nonanedioate) | LCneg | 0.0478 | 118.3270 |
| 3141 | betaine | LCpos | 0.0093 | 91.2635 |
| 569 | caffeine | LCpos | 0.0179 | −70.6204 |
| 15506 | choline | LCpos | 0.0016 | 45.0093 |
| 12025 | cis-aconitate | LCpos | 0.0364 | 22.2510 |
| 22158 | citramalate | GC | 4.00E−04 | 59.4381 |
| 1564 | citrate | GC | 0.0019 | 139.2617 |
| 2132 | citrulline | GC | 4.00E−04 | 93.6606 |
| 27718 | creatine | LCpos | 4.00E−04 | 43.7043 |
| 20700 | cyanurate | GC | 0.0139 | 0.0000 |
| 31454 | cystine | GC | 0.0026 | 170.2201 |
| 32425 | dehydroisoandrosterone sulfate (DHEA-S) | LCneg | 0.0291 | 162.9464 |
| 15743 | dimethylarginine | LCpos | 2.00E−04 | 42.3710 |
| 5086 | dimethylglycine | GC | 0.0294 | 105.5877 |
| 32511 | EDTA | LCneg | 0.005 | −10.4294 |
| 20699 | erythritol | GC | 2.45E−05 | 54.8561 |
| 33477 | Erythronate | GC | 3.10E−05 | 34.5359 |
| 577 | Fructose | GC | 0.0373 | 152.8917 |
| 1643 | Fumarate | GC | 3.81E−05 | 61.1976 |
| 1117 | galactitol-dulcitol- | GC | 0.049 | −30.9639 |
| 34456 | gamma-glutamylisoleucine | LCpos | 0.0032 | 12.7695 |
| 18369 | gamma-glutamylleucine | LCpos | 5.00E−04 | 202.0740 |
| 33422 | gammaglutamylphenylalanine | LCpos | 0.0013 | 170.8455 |
| 2734 | gamma-glutamyltyrosine | LCpos | 6.00E−04 | 199.6524 |
| 18280 | gentisate | LCneg | 0.0254 | 84.1857 |
| 1476 | glucarate (saccharate) | GC | 0.0163 | 93.0656 |
| 587 | gluconate | GC | 1.00E−04 | 49.6957 |
| 18534 | glucosamine | GC | 1.00E−04 | 56.1753 |
| 20488 | glucose | GC | 1.00E−04 | 57.0890 |
| 15443 | glucuronate | GC | 6.00E−04 | 49.1315 |
| 57 | glutamate | GC | 0.0332 | 15.2177 |
| 32393 | glutamylvaline | LCpos | 7.00E−04 | 82.6082 |
| 15990 | glycerophosphorylcholine (GPC) | LCpos | 0.0092 | 22.5740 |
| 11777 | glycineQUM | GC | 0.01 | 47.6937 |
| 15737 | glycolate (hydroxyacetate) | GC | 0.0125 | 115.3677 |
| 22171 | glycylproline | LCpos | 0.0156 | 64.5671 |
| 12359 | guanidinoacetate | GC | 3.00E−04 | 186.4843 |
| 418 | guanine | GC | 0.0129 | 80.4718 |
| 33454 | gulono-1-4-lactone | GC | 5.00E−04 | 39.8172 |
| 15753 | hippurate | LCpos | 0.032 | 50.4495 |
| 1101 | homovanillate (HVA) | GC | 0.0044 | 34.8863 |
| 3127 | hypoxanthine | LCpos | 0.0266 | 25.2729 |
| 15716 | imidazole lactate | LCpos | 4.00E−04 | 47.0735 |
| 33846 | indoleacetate | LCpos | 0.0345 | 88.8776 |
| 18349 | indolelactate | GC | 0.0038 | 132.9586 |
| 33441 | isobutyrylcarnitine | LCpos | 0.0017 | 75.8028 |
| 1125 | isoleucine | LCpos | 0.0036 | 27.0710 |
| 34407 | isovalerylcarnitine | LCpos | 0.0046 | 42.2654 |
| 1417 | kynurenate | LCneg | 0.025 | 39.6023 |
| 15140 | kynurenine | LCpos | 0.0095 | 141.9643 |
| 11454 | lactose | GC | 0.0075 | 125.7434 |
| 60 | leucine | LCpos | 0.0088 | 26.6660 |
| 584 | mannose | GC | 0.0294 | 177.4984 |
| 18493 | mesaconate (methylfumarate) | GC | 0.008 | 85.1195 |
| 1302 | methionine | GC | 0.002 | 64.4250 |
| 34285 | monoethanolamine | GC | 0.0024 | 52.3196 |
| 33953 | N-acetylarginine | LCneg | 0.0014 | 116.6228 |
| 33942 | N-acetylasparagine | LCpos | 0.0134 | 79.3354 |
| 32195 | N-acetylaspartate (NAA) | GC | 0.0011 | 69.7707 |
| 15720 | N-acetylglutamate | LCpos | 0.009 | 41.1751 |
| 33943 | N-acetylglutamine | LCneg | 0.0294 | 65.6816 |
| 33946 | N-acetylhistidine | LCneg | 0.0046 | 81.9682 |
| 33967 | N-acetylisoleucine | LCpos | 0.0055 | 36.8144 |
| 1587 | N-acetylleucine | LCpos | 0.0042 | 107.1016 |
| 1592 | N-acetylneuraminate | GC | 0.0028 | 149.4873 |
| 33950 | N-acetylphenylalanine | LCpos | 0.0012 | 76.0267 |
| 33939 | N-acetylthreonine | LCpos | 0.026 | 89.8599 |
| 32390 | N-acetyltyrosine | LCpos | 3.00E−04 | 148.0601 |
| 1591 | N-acetylvaline | GC | 0.0035 | 148.2682 |
| 31850 | N-butyrylglycine | LCneg | 0.0356 | 46.9738 |
| 1598 | N-tigloylglycine | LCpos | 0.0186 | 36.7886 |
| 33936 | octanoylcarnitine | LCpos | 0.0063 | 32.2576 |
| 1505 | orotate | GC | 1.00E−04 | 57.3419 |
| 32558 | p-cresol sulfate | LCneg | 0.0203 | 67.1842 |
| 32718 | phenylacetylglutamine- | LCpos | 0.0177 | 42.1472 |
| 33945 | phenylacetylglycine | LCpos | 0.0049 | 102.7455 |
| 64 | phenylalanine | LCpos | 0.0137 | 70.3716 |
| 11438 | phosphate | GC | 0.0112 | 66.4883 |
| 1512 | picolinate | GC | 0.0401 | 23.7291 |
| 1898 | proline | GC | 0.0084 | 49.8421 |
| 33442 | pseudouridine | LCpos | 0.0069 | 18.3476 |
| 1651 | pyridoxal | LCpos | 0.0212 | 77.6885 |
| 599 | pyruvate | GC | 0.0104 | 68.1170 |
| 18335 | quinate | GC | 0.0412 | 40.7535 |
| 1899 | quinolinate | LCpos | 0.0068 | 81.2769 |
| 27731 | ribonate | GC | 4.00E−04 | 61.5332 |
| 15948 | S-adenosylhomocysteine (SAH) | LCpos | 0.0108 | 84.3170 |
| 1516 | sarcosineQUM | GC | 0.0073 | 103.7037 |
| 32379 | scyllo-inositol | GC | 0.0435 | 154.8068 |
| 1648 | serine | GC | 3.00E−04 | 49.1580 |
| 485 | spermidine | LCpos | 0.0459 | −81.3755 |
| 2125 | taurine | GC | 0.0334 | 172.8511 |
| 12360 | tetrahydrobiopterin | GC | 0.0116 | 69.2047 |
| 27738 | threonate | GC | 0.0012 | 51.7428 |
| 1284 | threonine | GC | 0.0056 | 139.5883 |
| 604 | thymine | GC | 0.0034 | 161.2888 |
| 6104 | tryptamine | LCpos | 0.0372 | 62.1316 |
| 54 | tryptophan | LCpos | 0.0091 | 70.7395 |
| 1603 | tyramine | LCpos | 0.0493 | 35.8870 |
| 1299 | tyrosine | GC | 0.0011 | 58.4261 |
| 605 | uracil | GC | 0.0015 | 129.5276 |
| 607 | urocanate | LCpos | 0.0072 | 68.0070 |
| 34406 | valerylcarnitine | LCpos | 0.0306 | 120.0406 |
| 1649 | valine | LCpos | 2.00E−04 | 54.9329 |
| 1567 | vanillylmandelate-VMA- | LCneg | 0.0443 | 49.0489 |
| 3147 | xanthine | LCpos | 0.0331 | 44.5844 |
| 15136 | xanthosine | LCpos | 0.0156 | 85.5165 |
| 15679 | xanthurenate | LCpos | 0.0077 | 27.7713 |
| 15835 | xylose | GC | 0.0137 | 81.6462 |
| 32735 | X-01911_200 | LCpos | 0.0143 | 234.5459 |
| 33009 | X-01981_200 | LCpos | 0.0017 | 48.0588 |
| 32550 | X-02272_201 | LCneg | 0.0247 | 51.0244 |
| 32672 | X-02546_200 | LCpos | 5.00E−04 | 79.4250 |
| 32709 | X-03056_200 | LCpos | 0.0142 | 15.1147 |
| 32653 | X-03249_200 | LCpos | 0.0051 | 100.7635 |
| 32675 | X-03951_200 | LCpos | 6.00E−04 | 22.8452 |
| 32937 | X-03951_201 | LCneg | 4.00E−04 | 27.1295 |
| 32557 | X-06126_201 | LCneg | 0.023 | 106.4585 |
| 24332 | X-10128 | GC | 2.00E−04 | 52.5090 |
| 24469 | X-10266 | GC | 0.0032 | 38.3625 |
| 25401 | X-10359 | GC | 0.0024 | 33.6027 |
| 25402 | X-10360 | GC | 0.0262 | 44.6591 |
| 25449 | X-10385 | GC | 0.0136 | 49.8885 |
| 25607 | X-10437 | GC | 0.0474 | 86.7596 |
| 33014 | X-10457_200 | LCpos | 0.0476 | 22.6361 |
| 27883 | X-10604 | GC | 0.0077 | 43.5902 |
| 27884 | X-10605 | GC | 3.00E−04 | 40.8850 |
| 30275 | X-10738 | GC | 0.0049 | 55.5093 |
| 30276 | X-10739 | GC | 0.0034 | 82.2508 |
| 31022 | X-10831 | GC | 7.00E−04 | 67.9439 |
| 31041 | X-10835 | GC | 0.0051 | 108.0205 |
| 31053 | X-10841 | GC | 0.007 | 66.8101 |
| 31203 | X-10850 | GC | 0.0224 | 96.3934 |
| 31489 | X-10914 | GC | 0.0041 | 33.6270 |
| 31750 | X-11011 | GC | 1.00E−04 | 51.1781 |
| 31751 | X-11012 | GC | 1.00E−04 | 42.1647 |
| 31754 | X-11015 | GC | 0.002 | 43.7399 |
| 31757 | X-11018 | GC | 0.0188 | 209.6372 |
| 32026 | X-11072 | GC | 0.038 | 167.5549 |
| 32120 | X-11096 | GC | 0.0025 | 258.5659 |
| 32127 | X-11103 | GC | 0.026 | 288.9233 |
| 32562 | X-11245 | LCneg | 0.0419 | 116.4416 |
| 32578 | X-11261 | LCpos | 0.0357 | 53.5881 |
| 32599 | X-11282 | LCneg | 0.0211 | 124.6693 |
| 32649 | X-11332 | LCpos | 0.0303 | −41.3196 |
| 32650 | X-11333 | LCpos | 0.0359 | 53.6853 |
| 32664 | X-11347 | LCpos | 1.00E−04 | 30.8069 |
| 32665 | X-11348_200 | LCpos | 6.00E−04 | 37.7556 |
| 32669 | X-11352 | LCpos | 0.0163 | 51.3693 |
| 32674 | X-11357 | LCpos | 0.0314 | 55.2106 |
| 32714 | X-11397 | LCpos | 0.038 | 126.7154 |
| 32738 | X-11421 | LCpos | 0.0318 | 69.8841 |
| 32740 | X-11423 | LCneg | 0.0151 | 15.7989 |
| 32761 | X-11444 | LCneg | 3.00E−04 | 33.3214 |
| 32767 | X-11450 | LCneg | 0.0461 | 86.9345 |
| 32769 | X-11452 | LCneg | 0.0055 | 95.2700 |
| 32781 | X-11464 | LCpos | 0.0435 | 53.2915 |
| 32787 | X-11470 | LCneg | 0.027 | 13.3518 |
| 32792 | X-11475 | LCneg | 0.0032 | 292.2009 |
| 32807 | X-11490 | LCneg | 0.0092 | 91.7365 |
| 32881 | X-11564 | LCneg | 8.00E−04 | 31.9184 |
| 32910 | X-11593 | LCneg | 0.0435 | 45.1354 |
| 32957 | X-11640 | LCneg | 0.0209 | 111.1731 |
| 32996 | X-11668 | LCneg | 0.0196 | 39.8008 |
| 33031 | X-11687 | LCpos | 0.0016 | 27.7502 |
| 33033 | X-11689 | LCpos | 0.0199 | 46.8620 |
| 33090 | X-11745 | GC | 0.0318 | 35.4414 |
| 33094 | X-11749 | GC | 0.0082 | 63.4649 |
| 33100 | X-11755 | GC | 0.0023 | 48.7368 |
| 33103 | X-11758 | GC | 0.0157 | 30.5194 |
| 33106 | X-11761 | GC | 0.0034 | 61.6069 |
| 33127 | X-11782 | GC | 0.0083 | 314.9654 |
| 33171 | X-11826 | LCneg | 0.0042 | 178.7640 |
| 33188 | X-11843 | LCneg | 0.0076 | 460.0511 |
| 33195 | X-11850 | LCneg | 0.0394 | 210.3870 |
| 33280 | X-11935 | LCpos | 0.0016 | 19.1957 |
| 33281 | X-11936 | LCpos | 0.0151 | 12.3351 |
| 33290 | X-11945 | LCpos | 0.0012 | 32.5289 |
| 33291 | X-11946 | LCpos | 0.0439 | 90.4452 |
| 33325 | X-11979 | LCpos | 0.0052 | 22.8598 |
| 33347 | X-12001 | LCneg | 0.0019 | 170.7811 |
| 33352 | X-12006 | LCneg | 2.00E−04 | 25.9733 |
| 33356 | X-12010 | LCneg | 0.0078 | 72.4838 |
| 33359 | X-12013 | LCneg | 0.022 | 405.5324 |
| 33393 | X-12042 | LCneg | 0.0095 | 93.4761 |
| 33398 | X-12047 | LCpos | 0.0046 | 48.5667 |
| 33405 | X-12053 | LCpos | 0.0276 | 70.0004 |
| 33511 | X-12096 | LCpos | 0.0266 | 38.6810 |
| 33512 | X-12097 | LCpos | 0.0333 | 58.4217 |
| 33514 | X-12099 | LCpos | 0.0072 | 47.4618 |
| 33515 | X-12100 | LCpos | 0.0089 | 21.6757 |
| 33516 | X-12101 | LCpos | 1.00E−04 | 83.2818 |
| 33519 | X-12104 | LCpos | 0.0177 | 11.4120 |
| 33523 | X-12108 | LCpos | 0.026 | 44.2185 |
| 33528 | X-12113 | LCpos | 0.025 | 146.1043 |
| 33532 | X-12117 | LCpos | 0.0483 | 21.8348 |
| 33537 | X-12122 | LCpos | 0.0029 | 66.5031 |
| 33539 | X-12124 | LCpos | 9.00E−04 | 29.0229 |
| 33542 | X-12127 | LCpos | 0.0068 | 123.3782 |
| 33543 | X-12128 | LCpos | 0.0167 | 43.0535 |
| 33546 | X-12131 | LCpos | 0.0086 | 0.0000 |
| 33590 | X-12170_200 | LCpos | 0.003 | 23.1150 |
| 33594 | X-12173 | LCpos | 0.0417 | −52.8764 |
| 33609 | X-12188 | LCneg | 0.0277 | 80.8620 |
| 33614 | X-12193 | LCpos | 0.0114 | 140.4048 |
| 33620 | X-12199 | LCpos | 0.0109 | 195.2826 |
| 33627 | X-12206 | LCneg | 0.0095 | 15.5730 |
| 33632 | X-12211 | LCneg | 0.0038 | 217.1225 |
| 33633 | X-12212 | LCneg | 0.0361 | 220.1253 |
| 33638 | X-12217 | LCneg | 0.0266 | 42.5603 |
| 33646 | X-12225 | LCpos | 6.00E−04 | 20.7575 |
| 33658 | X-12236 | LCneg | 0.0258 | 109.4350 |
| 33669 | X-12247 | LCneg | 0.0156 | 38.0283 |
| 33676 | X-12254 | LCneg | 0.0315 | 229.5867 |
| 33683 | X-12261 | LCneg | 0.0224 | 215.2098 |
| 33704 | X-12282 | LCpos | 0.0032 | 78.5452 |
| 33728 | X-12306 | LCneg | 0.0356 | 115.0007 |
| 33745 | X-12323 | LCneg | 0.0191 | 36.7940 |
| 33764 | X-12339 | LCpos | 0.023 | 50.4166 |
| 33765 | X-12340 | LCpos | 0.0386 | 131.2436 |
| 33786 | X-12358 | LCpos | 0.0019 | 39.9305 |
| 33787 | X-12359 | LCpos | 0.0022 | 108.4776 |
| 33792 | X-12364 | LCpos | 0.015 | 52.5728 |
| 33804 | X-12376 | LCpos | 0.0037 | 52.2176 |
| 33807 | X-12379 | LCpos | 0.0335 | 84.0021 |
| 33814 | X-12386 | LCneg | 0.0028 | 79.8037 |
| 33835 | X-12407 | LCneg | 0.0419 | 102.2921 |
| 33839 | X-12411 | LCneg | 0.0469 | 181.1927 |
| 33903 | X-12458 | LCpos | 0.0454 | 3.8204 |
| 34041 | X-12511 | LCpos | 0.014 | 67.0961 |
| 34094 | X-12534 | GC | 0.0114 | 23.0764 |
| 34123 | X-12556 | GC | 0.0014 | 38.9741 |
| 34124 | X-12557 | GC | 0.0069 | 133.5437 |
| 34137 | X-12570 | GC | 6.00E−04 | 23.4172 |
| 34146 | X-12579 | GC | 0.0166 | 36.6870 |
| 34197 | X-12603 | LCneg | 0.0486 | 93.9915 |
| 34200 | X-12606 | LCneg | 0.0239 | 84.7583 |
| 34205 | X-12611 | LCpos | 0.0024 | 36.6540 |
| 34206 | X-12612 | LCpos | 0.0403 | 100.6866 |
| 34223 | X-12629 | LCpos | 0.0228 | 64.2063 |
| 34229 | X-12632 | LCpos | 0.0345 | 65.5474 |
| 34231 | X-12634 | LCpos | 0.0339 | 74.2212 |
| 34235 | X-12636 | LCpos | 0.0113 | 30.6322 |
| 34253 | X-12650 | LCpos | 0.0228 | 70.5815 |
| 34268 | X-12663 | GC | 0.0186 | 149.0884 |
| 34289 | X-12680 | LCpos | 0.0249 | 116.7362 |
| 34290 | X-12681 | LCpos | 0.0345 | 53.3469 |
| 34291 | X-12682 | LCpos | 0.0266 | 25.1312 |
| 34292 | X-12683 | LCpos | 0.0025 | 36.9150 |
| 34294 | X-12685 | LCpos | 0.0474 | 70.8178 |
| 34295 | X-12686 | LCpos | 0.0052 | 15.6282 |
| 34297 | X-12688 | LCpos | 0.0029 | 124.9182 |
| 34298 | X-12689 | LCpos | 0.0256 | 20.8243 |
| 34299 | X-12690 | LCpos | 0.0019 | 16.8796 |
| 34300 | X-12691 | LCpos | 0.016 | 81.0894 |
| 34304 | X-12694 | LCneg | 0.0292 | 30.3117 |
| 34305 | X-12695 | LCneg | 0.0083 | 51.2191 |
| 34310 | X-12700 | LCneg | 0.005 | 85.1265 |
| 34311 | X-12701 | LCneg | 0.0451 | 63.6861 |
| 34314 | X-12704 | LCneg | 0.0252 | 243.6844 |
| 34316 | X-12706 | LCneg | 0.0413 | 156.8494 |
| 34318 | X-12708 | LCneg | 0.015 | 79.9730 |
| 34322 | X-12712 | LCneg | 0.0487 | 79.2438 |
| 34325 | X-12715 | LCneg | 0.0049 | 55.2094 |
| 34327 | X-12717 | LCneg | 0.012 | 203.4073 |
| 34336 | X-12726 | LCneg | 0.0146 | 66.2239 |
| 34339 | X-12729 | LCneg | 0.0299 | 117.3626 |
| 34343 | X-12733 | LCneg | 0.0108 | 43.8603 |
| 34349 | X-12739 | LCneg | 0.0014 | 89.0934 |
| 34350 | X-12740 | LCneg | 0.0282 | 405.1284 |
| 34352 | X-12742 | LCneg | 0.0199 | 70.2457 |
| 34353 | X-12743 | LCneg | 6.38E−06 | 70.0243 |
| 34355 | X-12745 | LCneg | 0.0045 | 1230.4546 |
| 34358 | X-12748 | LCpos | 1.09E−05 | 68.9382 |
| 34359 | X-12749 | LCpos | 0.0196 | 14.6434 |
| 34360 | X-12750 | LCpos | 0.0452 | 34.9301 |
| 34362 | X-12752 | LCpos | 0.002 | 28.4767 |
| 34370 | X-12760 | LCpos | 0.007 | 41.6076 |
| 34375 | X-12765 | LCpos | 0.0016 | 57.1255 |
| 34485 | X-12802 | LCpos | 0.0031 | 47.2186 |
| 34497 | X-12814 | LCneg | 0.0349 | 216.9783 |
| 34498 | X-12815 | LCneg | 0.0497 | 98.1436 |
| 34503 | X-12820 | LCneg | 0.0467 | 348.8805 |
| 34505 | X-12822 | LCneg | 0.012 | 64.5382 |
| 34511 | X-12828 | LCneg | 0.0107 | 74.3241 |
| 34524 | X-12841 | LCneg | 0.0049 | 165.1258 |
| 34526 | X-12843 | LCneg | 0.0018 | 432.1185 |
| 34527 | X-12844 | LCneg | 0.0029 | 30.9475 |
| 34528 | X-12845 | LCneg | 0.0161 | 162.3770 |
| 34529 | X-12846 | LCneg | 0.0306 | 27.5410 |
| 34530 | X-12847 | LCneg | 0.0306 | 254.3334 |
| 34531 | X-12848 | LCneg | 0.0147 | 259.3802 |
| 34532 | X-12849 | LCneg | 0.022 | 232.6990 |
| 34533 | X-12850 | LCneg | 0.0106 | 152.3123 |
| 12603 | X-2980 | GC | 0.0435 | 150.0623 |
| 12770 | X-3090 | GC | 0.047 | 49.3716 |
| 16062 | X-4015 | GC | 5.00E−04 | 97.5835 |
| 16821 | X-4498 | GC | 5.00E−04 | 59.0953 |
| 16822 | X-4499 | GC | 2.00E−04 | 65.9952 |
| 16829 | X-4503 | GC | 0.0389 | 448.9493 |
| 16831 | X-4504 | GC | 0.0017 | 34.7506 |
| 16837 | X-4507 | GC | 0.0104 | 33.7584 |
| 16866 | X-4523 | GC | 2.00E−04 | 163.4988 |
| 16984 | X-4599 | GC | 0.0033 | 76.7293 |
| 17050 | X-4618 | GC | 0.0085 | 32.9874 |
| 17064 | X-4624 | GC | 0.0052 | 55.2961 |
| 17072 | X-4628 | GC | 0.0075 | 272.1564 |
| 17074 | X-4629 | GC | 1.00E−04 | 57.5233 |
| 17086 | X-4637 | GC | 6.00E−04 | 181.6876 |
| 17088 | X-4639 | GC | 0.0064 | 88.5308 |
| 18232 | X-5403 | GC | 0.0032 | 32.1164 |
| 18251 | X-5409 | GC | 0.0042 | 39.1551 |
| 18253 | X-5410 | GC | 0.017 | 355.5448 |
| 18257 | X-5412 | GC | 0.0104 | 48.5322 |
| 18264 | X-5414 | GC | 0.0032 | 135.2663 |
| 18265 | X-5415 | GC | 0.0171 | 40.2508 |
| 18271 | X-5418 | GC | 3.00E−04 | 65.0484 |
| 18272 | X-5419 | GC | 0.0082 | 49.3174 |
| 18273 | X-5420 | GC | 2.00E−04 | 50.7034 |
| 18307 | X-5431 | GC | 0.0046 | 267.5213 |
| 18309 | X-5433 | GC | 0.0094 | 131.5460 |
| 18316 | X-5437 | GC | 0.0075 | 142.7695 |
| 18388 | X-5491 | GC | 4.19E−05 | 58.3225 |
| 18390 | X-5492 | GC | 8.00E−04 | 46.4359 |
| 18419 | X-5506 | GC | 0.027 | 65.4907 |
| 18430 | X-5511 | GC | 0.0199 | 107.8683 |
| 18438 | X-5518 | GC | 0.0117 | 1692.6298 |
| 18442 | X-5522 | GC | 0.002 | 45.8239 |
| 19954 | X-6906 | GC | 1.00E−04 | 34.3189 |
| 19960 | X-6912 | GC | 0.0031 | 36.2744 |
| 19965 | X-6928 | GC | 0.0191 | 38.2332 |
| 19969 | X-6931 | GC | 0.0136 | 225.7159 |
| 19973 | X-6946 | GC | 0.003 | 126.2096 |
| 19984 | X-6956 | GC | 4.00E−04 | 77.8832 |
| 19990 | X-6962 | GC | 0.0149 | 42.7975 |
| 19997 | X-6969 | GC | 0.0037 | 545.8663 |
| 20014 | X-6985 | GC | 0.0474 | 106.4077 |
| 20020 | X-6991 | GC | 0.015 | 49.2941 |
| 22308 | X-8886 | GC | 0.0452 | 118.3757 |
| 22494 | X-8994 | GC | 0.017 | 567.8661 |
| 22548 | X-9026 | GC | 0.002 | 125.0265 |
| 22570 | X-9033 | GC | 0.0329 | 85.2545 |
| 22881 | X-9287 | GC | 0.0101 | 85.5217 |
| 24074 | X-9706 | GC | 0.0042 | 46.6887 |
| 24076 | X-9726 | GC | 0.0331 | 50.6677 |

The cancer status (e.g., non-cancer or cancer) of individual subjects was determined using the biomarkers sarcosine and N-acetyl tyrosine. Using these two markers in combination resulted in cancer diagnosis with 83% sensitivity and 49% specificity. Assuming a 30% prevalence of cancer in a PSA positive population, these biomarkers gave a Negative Predictive Value (NPV) of 87% and a Positive Predictive Value (PPV) of 41%.

Biomarkers that Distinguish Less Aggressive Cancer from More Aggressive Cancer:

The urine samples used for the analysis were obtained from individuals diagnosed with prostate cancer having biopsy scores of GS major 3 or GS major 4 and above. GSmajor3 indicates a lower grade of cancer that is typically less aggressive while GS major 4 indicates a higher grade of cancer that is typically more aggressive. In this analysis the GS major 3 subjects (N=45) were compared to subjects with a GS major 4 (N=13). After the levels of metabolites were determined, the data was analyzed using the Wilcoxon test to determine differences in the mean levels of metabolites between two populations (e.g., Prostate cancer vs. Control).

As listed below in Table 2, biomarkers were discovered that were differentially present between urine samples from subjects with less aggressive/lower grade prostate cancer and subjects with more aggressive/higher grade prostate cancer.

Table 2 includes, for each listed biomarker, the p-value determined in the statistical analysis of the data concerning the biomarkers, the compound ID useful to track the compound in the chemical database and the analytical platform used to identify the compounds (GC refers to GC/MS and LC refers to UHPLC/MS/MS2). P-values that are listed as 0.000 are significant at p<0.0001. LCpos and LCneg refer to UHPLC separation using buffers and parameters that are optimized for detecting positive ions or negative ions, respectively.

TABLE 2
Biomarkers that distinguish less aggressive from more aggressive prostate cancer.

| | | | | % Change in |
|---|---|---|---|---|
| COMP_ID | COMPOUND | Platform | p-value | Aggressive PCA |
| 34404 | 1,3-7-trimethyluric acid | LCneg | 0.0057 | −66.55113998 |
| 34400 | 1-7-dimethylurate | LCneg | 0.001 | −62.28917254 |
| 15650 | 1-methyladenosine | LCpos | 0.0254 | 43.02217774 |
| 34395 | 1-methylurate | LCpos | 4.00E−04 | −49.79665561 |
| 34389 | 1-methylxanthine | LCpos | 0.0138 | −67.90592259 |
| 15667 | 2-isopropylmalate | LCneg | 0.0469 | 166.2876883 |
| 18296 | 3-4-dihydroxyphenylacetate | GC | 0.0014 | 123.2216303 |
| 27672 | 3-indoxyl-sulfate | LCneg | 0.0138 | −23.7469546 |
| 12017 | 3-methoxytyrosine | LCpos | 0.0113 | 86.24357623 |
| 15677 | 3-methylhistidine | LCneg | 0.0059 | 102.3968054 |
| 32445 | 3-methylxanthine | LCpos | 0.0132 | −72.50497601 |
| 3155 | 3-ureidopropionate | LCpos | 0.022 | 27.56547555 |
| 1558 | 4-acetamidobutanoate | LCpos | 0.0166 | 59.98174305 |
| 15681 | 4-guanidinobutanoate | LCpos | 0.0297 | 174.6765122 |
| 21133 | 4-hydroxybenzoate | GC | 0.01 | 71.09064956 |
| 1568 | 4-hydroxymandelate | GC | 0.0208 | 89.80468995 |
| 22118 | 4-ureidobutyrate | LCpos | 0.017 | 60.30878737 |
| 437 | 5-hydroxyindoleacetate | GC | 0.0226 | 84.94805375 |
| 1494 | 5-oxoproline | LCpos | 0.0056 | −29.70497615 |
| 31580 | 7-methylguanosine | GC | 0.0347 | 84.95194026 |
| 555 | adenosine | LCpos | 0.0111 | 79.86819651 |
| 2831 | adenosine-3′,5′-cyclic- | LCpos | 0.0136 | 53.42430461 |
| | monophosphate (cAMP) | | | |
| 15142 | allo-threonine | GC | 5.00E−04 | 307.6014316 |
| 575 | arabinose | GC | 0.0079 | 148.4557 |
| 15964 | arabitol | GC | 0.0441 | 98.60829547 |
| 1640 | ascorbate (Vitamin C) | GC | 0.045 | 175.9986664 |
| 18362 | azelate (nonanedioate) | LCneg | 0.0186 | 207.3082051 |
| 3141 | betaineQUM | LCpos | 0.0019 | 111.1077205 |
| 569 | caffeine | LCpos | 0.0075 | −81.71522011 |
| 12025 | cis-aconitate | LCpos | 0.0369 | −25.83372809 |
| 1564 | citrate | GC | 0.0153 | 159.3164801 |
| 27718 | creatine | LCpos | 0.0062 | 239.6294824 |
| 513 | creatinine | LCpos | 0.0291 | 77.95100223 |
| 32425 | dehydroisoandrosterone sulfate | LCneg | 0.0272 | 153.7895042 |
| | (DHEA-S) | | | |
| 5086 | dimethylglycine | GC | 0.0084 | 89.87003058 |
| 1643 | fumarate | GC | 0.023 | −27.15601216 |
| 1117 | galactitol-dulcitol- | GC | 0.0036 | 352.7349757 |
| 34456 | gamma-glutamylisoleucine* | LCpos | 0.0198 | 83.47303345 |
| 18369 | gamma-glutamylleucine | LCpos | 8.00E−04 | 100.8835487 |
| 33422 | gammaglutamylphenylalanine | LCpos | 8.00E−04 | 116.4623197 |
| 2734 | gamma-glutamyltyrosine | LCpos | 0.0018 | 199.6523546 |
| 1476 | glucarate (saccharate) | GC | 0.0413 | 78.73546464 |
| 587 | gluconate | GC | 0.0337 | 135.3595762 |
| 15443 | glucuronate | GC | 0.048 | 79.98123372 |
| 32393 | glutamylvaline | LCpos | 0.005 | 53.61399238 |
| 15365 | glycerol 3-phosphate (G3P) | GC | 0.0095 | 96.65755153 |
| 15990 | glycerophosphorylcholine (GPC) | LCpos | 0.043 | −30.99560024 |
| 11777 | glycine | GC | 0.0047 | 51.51603573 |
| 15737 | glycolate (hydroxyacetate) | GC | 0.0219 | 103.7720467 |
| 22171 | glycylproline | LCpos | 0.0081 | 81.31832313 |
| 12359 | guanidinoacetate | GC | 0.0015 | 163.1261154 |
| 33454 | gulono-1-4-lactone | GC | 0.0413 | 61.59491649 |
| 1101 | homovanillate (HVA) | GC | 0.0081 | 87.32242401 |
| 21025 | iminodiacetate-IDA- | GC | 0.021 | 44.48398584 |
| 33846 | indoleacetate | LCpos | 0.0362 | 105.8783175 |
| 18349 | indolelactate | GC | 0.0332 | 101.7860312 |
| 33441 | isobutyrylcarnitine | LCpos | 0.0279 | 55.35226019 |
| 12110 | isocitrate | LCpos | 0.0422 | −41.41198939 |
| 1125 | isoleucine | LCpos | 0.0208 | 54.70179416 |
| 15140 | kynurenine | LCpos | 0.0191 | 132.392076 |
| 527 | lactate | GC | 0.0337 | −29.28603115 |
| 11454 | lactose | GC | 0.0117 | 108.8417975 |
| 60 | leucine | LCpos | 0.0332 | 44.16653491 |
| 584 | mannose | GC | 0.0158 | 108.0495974 |
| 18493 | mesaconate (methylfumarate) | GC | 0.0452 | −48.02028356 |
| 1302 | methionine | GC | 0.01 | 93.23111101 |
| 34285 | monoethanolamine | GC | 0.0363 | 159.4495524 |
| 33953 | N-acetylarginine | LCneg | 0.0317 | 85.9617038 |
| 32195 | N-acetylaspartate (NAA) | GC | 0.0379 | 94.62417064 |
| 33946 | N-acetylhistidine | LCneg | 0.0058 | 59.11465726 |
| 1587 | N-acetylleucine | LCpos | 0.0227 | 85.37871881 |
| 33950 | N-acetylphenylalanine | LCpos | 0.0095 | 66.64423652 |
| 33939 | N-acetylthreonine | LCpos | 0.0332 | 78.16412969 |
| 32390 | N-acetyltyrosine | LCpos | 0.0057 | 133.7952527 |
| 1591 | N-acetylvaline | GC | 0.0463 | 66.01491718 |
| 18254 | paraxanthine | LCpos | 0.0219 | −63.90495686 |
| 33945 | phenylacetylglycine | LCpos | 0.006 | 90.17463794 |
| 64 | phenylalanine | LCpos | 0.0254 | 57.32016167 |
| 33442 | pseudouridine | LCpos | 0.0231 | 54.52078056 |
| 1651 | pyridoxal | LCpos | 0.0268 | 54.86441025 |
| 599 | pyruvate | GC | 0.0071 | 62.1494331 |
| 1899 | quinolinate | LCpos | 0.006 | 61.91679621 |
| 27731 | ribonate | GC | 0.0394 | 100.3888599 |
| 15948 | S-adenosylhomocysteine (SAH) | LCpos | 0.0344 | 62.81234124 |
| 1516 | sarcosine | GC | 0.0021 | 89.65517241 |
| 1648 | serine | GC | 0.0337 | 80.59915169 |
| 603 | spermine | LCpos | 0.0247 | −78.26667362 |
| 18392 | theobromine | LCpos | 0.0165 | −80.1429027 |
| 27738 | threonate | GC | 0.0396 | 94.31081416 |
| 1284 | threonine | GC | 0.0118 | 77.88106938 |
| 604 | thymine | GC | 0.0157 | 71.13143504 |
| 54 | tryptophan | LCpos | 0.0162 | 80.30828074 |
| 1299 | tyrosine | GC | 0.008 | 99.33740457 |
| 605 | uracil | GC | 0.0318 | 75.86987921 |
| 32701 | urate- | LCpos | 0.0482 | −49.86065084 |
| 607 | urocanate | LCpos | 0.0219 | 55.53807526 |
| 1649 | valine | LCpos | 0.0266 | 132.4327688 |
| 15835 | xylose | GC | 0.0219 | 79.58039821 |
| 32672 | X-02546_200 | LCpos | 0.0124 | 39.92995063 |
| 32653 | X-03249_200 | LCpos | 0.0347 | 50.52155844 |
| 32675 | X-03951_200 | LCpos | 0.0461 | 77.31945011 |
| 32937 | X-03951_201 | LCneg | 0.0404 | 84.92252578 |
| 24469 | X-10266 | GC | 0.0276 | 73.92296217 |
| 25402 | X-10360 | GC | 0.0347 | 79.71371779 |
| 33014 | X-10457_200 | LCpos | 0.0369 | 26.87901527 |
| 27884 | X-10605 | GC | 0.0379 | 117.0583917 |
| 31751 | X-11012 | GC | 0.0266 | 126.3470402 |
| 31754 | X-11015 | GC | 0.0396 | 60.66427028 |
| 32026 | X-11072 | GC | 0.0204 | 111.0816308 |
| 32120 | X-11096 | GC | 0.002 | 246.5355958 |
| 32562 | X-11245 | LCneg | 0.022 | 147.5795427 |
| 32631 | X-11314 | LCpos | 0.0347 | −38.84300738 |
| 32649 | X-11332 | LCpos | 0.0059 | 104.0484707 |
| 32651 | X-11334 | LCpos | 0.0321 | 69.54121645 |
| 32652 | X-11335 | LCpos | 0.0379 | 65.56679429 |
| 32665 | X-11348_200 | LCpos | 0.0369 | 71.33451227 |
| 32714 | X-11397 | LCpos | 0.0277 | −67.48708723 |
| 32754 | X-11437 | LCneg | 0.0047 | 1257.122467 |
| 32767 | X-11450 | LCneg | 0.0363 | 79.38640823 |
| 32792 | X-11475 | LCneg | 0.0031 | 366.4908828 |
| 32807 | X-11490 | LCneg | 0.0466 | 84.13891831 |
| 32827 | X-11510 | LCneg | 0.015 | 137.5062988 |
| 32878 | X-11561 | LCneg | 0.0347 | 39.08827189 |
| 32978 | X-11656 | LCpos | 0.045 | −55.75256194 |
| 33171 | X-11826 | LCneg | 0.0064 | 144.2554847 |
| 33280 | X-11935 | LCpos | 0.0293 | 61.44828759 |
| 33281 | X-11936 | LCpos | 0.0266 | 53.18088504 |
| 33290 | X-11945 | LCpos | 0.0461 | 51.88262935 |
| 33291 | X-11946 | LCpos | 0.0433 | 57.82662663 |
| 33295 | X-11949 | LCpos | 0.0321 | −26.25001217 |
| 33325 | X-11979 | LCpos | 0.0278 | 48.01647625 |
| 33352 | X-12006 | LCneg | 0.0304 | 73.56750455 |
| 33356 | X-12010 | LCneg | 0.0083 | 233.0064131 |
| 33361 | X-12015 | LCneg | 0.0158 | 106.0732039 |
| 33393 | X-12042 | LCneg | 0.0173 | 74.91590711 |
| 33398 | X-12047 | LCpos | 0.0219 | 55.34246459 |
| 33514 | X-12099 | LCpos | 0.0129 | 47.01102723 |
| 33516 | X-12101 | LCpos | 0.0103 | −36.00760478 |
| 33530 | X-12115 | LCpos | 0.0441 | −33.02940864 |
| 33537 | X-12122 | LCpos | 0.0253 | 49.52870476 |
| 33539 | X-12124 | LCpos | 0.0347 | 46.14882349 |
| 33542 | X-12127 | LCpos | 0.0254 | 89.89660466 |
| 33543 | X-12128 | LCpos | 0.0034 | −55.28552444 |
| 33609 | X-12188 | LCneg | 0.0071 | −77.72107587 |
| 33614 | X-12193 | LCpos | 0.0063 | 116.7744629 |
| 33620 | X-12199 | LCpos | 0.0254 | 161.7656256 |
| 33632 | X-12211 | LCneg | 0.0216 | 203.3196007 |
| 33633 | X-12212 | LCneg | 0.033 | 280.5910199 |
| 33637 | X-12216 | LCneg | 0.0118 | −52.22252608 |
| 33638 | X-12217 | LCneg | 0.0482 | −39.44206727 |
| 33646 | X-12225 | LCpos | 0.0075 | 59.98551337 |
| 33665 | X-12243 | LCpos | 0.0253 | −47.60623384 |
| 33676 | X-12254 | LCneg | 0.0191 | 415.8798474 |
| 33704 | X-12282 | LCpos | 0.0059 | 58.42472716 |
| 33764 | X-12339 | LCpos | 0.0413 | 40.70759506 |
| 33774 | X-12349 | LCneg | 0.0198 | −25.18575014 |
| 33787 | X-12359 | LCpos | 0.0111 | 93.83073384 |
| 33804 | X-12376 | LCpos | 0.0124 | 58.66527499 |
| 33814 | X-12386 | LCneg | 0.0136 | 108.2300401 |
| 33835 | X-12407 | LCneg | 0.0489 | 55.24997178 |
| 33839 | X-12411 | LCneg | 0.019 | 87.92801957 |
| 33910 | X-12465 | LCpos | 0.0218 | 0 |
| 34041 | X-12511 | LCpos | 0.0179 | 89.02312659 |
| 34094 | X-12534 | GC | 0.0369 | 15.74666369 |
| 34123 | X-12556 | GC | 0.0386 | 55.12702293 |
| 34137 | X-12570 | GC | 0.029 | 72.94401006 |
| 34138 | X-12571 | LCpos | 0.0461 | −51.97060823 |
| 34170 | X-12602 | LCpos | 0.0327 | 33.15918309 |
| 34268 | X-12663 | GC | 0.0265 | 82.0191453 |
| 34289 | X-12680 | LCpos | 0.045 | 93.83428843 |
| 34290 | X-12681 | LCpos | 0.0431 | 67.59059032 |
| 34292 | X-12683 | LCpos | 0.0468 | 76.11571819 |
| 34294 | X-12685 | LCpos | 0.0128 | 114.0988325 |
| 34295 | X-12686 | LCpos | 0.0461 | 54.50094449 |
| 34297 | X-12688 | LCpos | 0.0084 | 100.1303934 |
| 34299 | X-12690 | LCpos | 0.0353 | 74.54432605 |
| 34300 | X-12691 | LCpos | 0.0325 | 67.30133053 |
| 34305 | X-12695 | LCneg | 0.0321 | 52.64061636 |
| 34310 | X-12700 | LCneg | 0.0073 | 102.1108558 |
| 34311 | X-12701 | LCneg | 0.0428 | 159.9798899 |
| 34322 | X-12712 | LCneg | 0.0362 | 107.510855 |
| 34323 | X-12713 | LCneg | 0.0253 | 141.1585404 |
| 34332 | X-12722 | LCneg | 0.0181 | 120.1175671 |
| 34339 | X-12729 | LCneg | 0.0428 | 210.5959332 |
| 34343 | X-12733 | LCneg | 0.0037 | −57.78309079 |
| 34349 | X-12739 | LCneg | 0.0198 | −37.87433792 |
| 34350 | X-12740 | LCneg | 0.0158 | 441.3133411 |
| 34352 | X-12742 | LCneg | 0.0307 | −48.53620833 |
| 34353 | X-12743 | LCneg | 0.0138 | 155.1605436 |
| 34355 | X-12745 | LCneg | 0.0354 | 471.2309818 |
| 34358 | X-12748 | LCpos | 0.0461 | −13.09684771 |
| 34359 | X-12749 | LCpos | 0.0242 | −23.31492948 |
| 34360 | X-12750 | LCpos | 0.0297 | 26.42009682 |
| 34372 | X-12762 | LCpos | 0.0412 | 178.3117468 |
| 34497 | X-12814 | LCneg | 0.04 | 170.9153319 |
| 34498 | X-12815 | LCneg | 0.0242 | 98.14355773 |
| 34505 | X-12822 | LCneg | 0.0325 | 43.0072576 |
| 34524 | X-12841 | LCneg | 0.0182 | 189.4742509 |
| 34526 | X-12843 | LCneg | 0.0066 | 118.568709 |
| 34528 | X-12845 | LCneg | 0.023 | 162.3770256 |
| 34532 | X-12849 | LCneg | 0.0143 | 173.837207 |
| 34533 | X-12850 | LCneg | 0.0233 | 138.2604803 |
| 12785 | X-3103 | GC | 0.0482 | −47.31496658 |
| 16062 | X-4015 | GC | 0.0037 | 43.60275909 |
| 16831 | X-4504 | GC | 0.0321 | 120.6164818 |
| 17086 | X-4637 | GC | 0.0028 | 281.0902182 |
| 18251 | X-5409 | GC | 0.0191 | 71.87489485 |
| 18264 | X-5414 | GC | 0.015 | 90.0100388 |
| 18265 | X-5415 | GC | 0.0413 | 101.7549199 |
| 18316 | X-5437 | GC | 0.0053 | 128.193364 |
| 18388 | X-5491 | GC | 0.023 | −31.91685364 |
| 19960 | X-6912 | GC | 0.0242 | 129.4486593 |
| 19965 | X-6928 | GC | 0.0317 | 125.0950831 |
| 19969 | X-6931 | GC | 0.0278 | 180.8662725 |
| 19973 | X-6946 | GC | 0.0061 | 149.537457 |
| 19990 | X-6962 | GC | 0.0413 | 34.36068338 |
| 19997 | X-6969 | GC | 0.0145 | 545.8663231 |
| 22320 | X-8889 | GC | 0.0441 | 41.201698 |
| 22494 | X-8994 | GC | 0.0236 | 805.8059769 |
| 22570 | X-9033 | GC | 0.0219 | −94.82653652 |
| 24074 | X-9706 | GC | 0.0482 | 35.47108011 |

### Example 2

### Validation of Multiple Metabolites in Prostate Cancer Tissues, Cell Lines, and Urine Sediment

### Materials and Methods:

Amino acids were obtained from Sigma (St. Louis, Mo., USA). Corresponding labeled versions were obtained from Isotec (Miamisburg, Ohio, USA). Isobutanol, choloroform, acetonitrile, dimethylformamide, ethylacetate were obtained from Sigma. All other chemical of analytical-reagent grade and obtained from Fluka and Sigma. N-methyl-N-(tert-butylmethylsilyltrifluoroacetamide (MtBSTFA)+1% t-butyl-dimethylchlorosilane and heptafluorobutyryl anhydride were purchased from Regis Technologies Inc, IL, USA.

### Clinical Samples

Benign prostate and localized prostate cancer tissues were obtained from a radical prostatectomy series at the University of Michigan Hospitals and the metastatic prostate cancer biospecimens were from the Rapid Autopsy Program, which are both part of University of Michigan Prostate Cancer Specialized Program of Research Excellence (S.P.O.R.E) Tissue Core. Samples were collected with informed consent and prior institutional review board approval at the University of Michigan. In addition, matched urine samples were collected post-DRE and prior to biopsy. This includes both biopsy-positive/negative patients as well as from patients with non-cancer-related prostate pathology. All samples were stored at −80° C. until use.

### Sample Preparation and Validation of Multiple Metabolites in Clinical Specimens Using GC-MS

Biological samples (Tissues, Urine and cell lines) were homogenized in methanol after spiking labeled internal standards and kept shaking for overnight at 4° C. The extraction was carried using 1:1 molar ratio of water/chloroform at room temperature for 30 minutes. The aqueous methanolic layer was collected and dried completely under nitrogen. The methonolic dried extract containing metabolites were further analyzed by GC-MS after derivatization using MtBSTFA. The dried methanolic amino acid residue, azeotrope twice by adding 100 μL dimethylformamide (DMF), vortexing, and then drying in a speed vac for 30 minutes. 100 μL of DMF and N-methyl-N-(tert-butylmethylsilyltrifluoroacetamide (MtBSTFA)+1% t-butyl-dimethylchlorosilane were added to the dried sample, capped, and incubated at 60° C. for 1 hr and then resuspended with ethyl acetate and injected into a GC-MS.

Selective Ion Monitoring (SIM) was used for quantification. The amount of metabolite in the sample was calculated by measuring the peak area of the native metabolite to the area of the peak for the isotope-labeled internal standard.

### Sample Preparation and Validation of Polyamines in Tissues by Gas Chromatography-Mass Spectrometry

Methanol was used to lyse tissues after spiking labeled internal standards and kept shaking overnight at 4° C. The extraction was carried out using a 1:1 molar ratio of water/chloroform at room temperature. The aqueous layer containing polyamines was collected and dried. The dried extract containing polyamines was further analyzed by GC-MS after derivatization using HFBA. To the dried residue, 200 μL of acetonitrile and 100 μL of HFBA were added. The vials were capped and heated at 65° C. for 60 min. The reaction mixture was then evaporated to dryness under a stream of nitrogen and then redissolved in 1 mL of diethyl ether. The ether solution was washed once with an equal volume of saturated sodium carbonate solution. After centrifugation, the aqueous phase was discarded and 1 μL of the ether phase taken for the GC-MS analysis. Selective Ion Monitoring (SIM) was used for quantification. The amount of polyamines in the sample was calculated by measuring the peak area of the native polyamines to the area of the peak for the isotope-labeled internal standard.

### Sample Preparation and Validation of Polyamines in Urine by Gas Chromatography-Mass Spectrometry

The isotope dilution GC/MS analysis of polyamines in urine used the modified method reported for sarcosine. The polyamines from biological samples were extracted by liquid-liquid extraction (MeOH:H₂O:CHCl₃, 1:1:1 ratio). The methanolic layer containing polyamines was evaporated to dryness under a stream of pure nitrogen. To the dried methanolic extract, 300 μL of isobutanol/3N HCl was added. The reaction mixture was introduced into a Pyrex test tube and closed with a screw cap covered by a Teflon septum. After heating the tube in a sand bath at 110° C. for 30 min, the samples were cooled and dried with a gentle nitrogen flow. Then, the samples were dried a second time after addition of 150 μL of acetonitrile to eliminate the residual moisture. Acetonitrile (200 μL) and 100 μL heptafluorobutyryl anhydride (HFBA) was added and the tube closed and heated at 125° C. for 20 min to generate heptafluorobutyle derivatives. The derivatized sample was analyzed by GC-MS. The polyamines were quantified using SIM analysis by measuring the peak areas of native polyamine to the area of the peak for the isotope-labeled internal standard. Methionine was used as an internal control for normalization of the urine concentration.

### Sample Preparation and Validation of Fatty Acids in Tissues Using Gas-Chromatography Mass Spectrometry:

Biological samples were homogenized in methanol after spiking labeled internal fatty acid standards and kept shaking for overnight at 4° C. The extraction was carried using 1:1 molar ratio of water/chloroform at room temperature for 30 minutes. The organic lipid layer was collected and dried completely under nitrogen. The chloroform dried extract containing metabolites (fatty acids) was further analyzed by GC-MS after derivatization using MtBSTFA. The dried organic fatty acid residue, azeotrope twice by adding 100 μL dimethylformamide (DMF), vortexing, and then drying in a speed vac for 30 minutes. 100 μL of DMF and N-methyl-N-(tert-butylmethylsilyltrifluoroacetamide (MtBSTFA)+1% t-butyl-dimethylchlorosilane was added to the dried sample, capped, and incubated at 60° C. for 1 hr and then resuspended with ethyl acetate and injected into a GC-MS.

### Results

### Development of Multiplex Panel Utilizing Prostate-Specific Metabolites

The elevated levels of sarcosine found in experiments conducted during the course of developing some embodiments of the present invention indicate that sarcosine is a prognostic marker for cancer (e.g., prostate cancer). In some embodiments of the present invention, tissue-derived, prostate cancer-specific find use as a multiplexed biomarker panel for the early detection of this disease.

### Validation of Multiple Metabolites in Tissues:

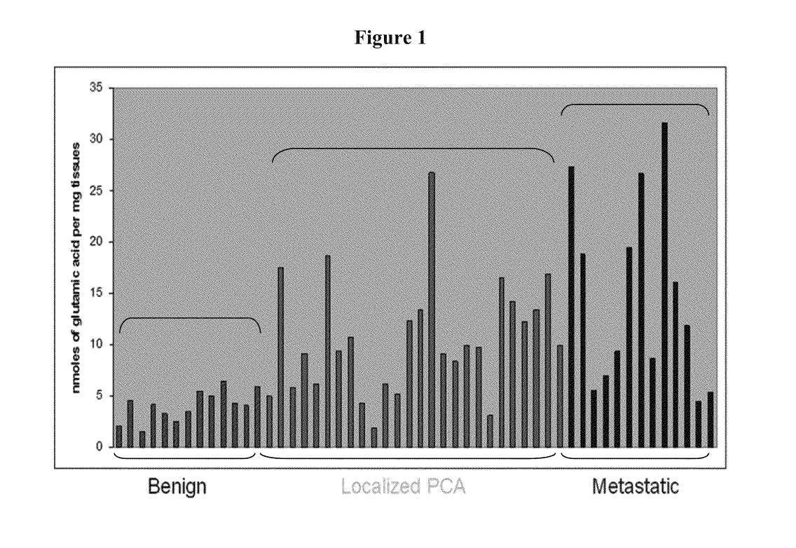

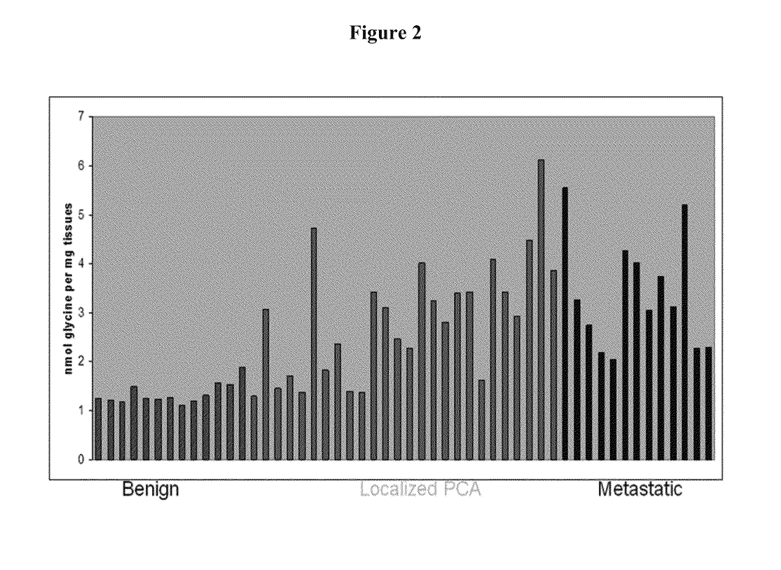

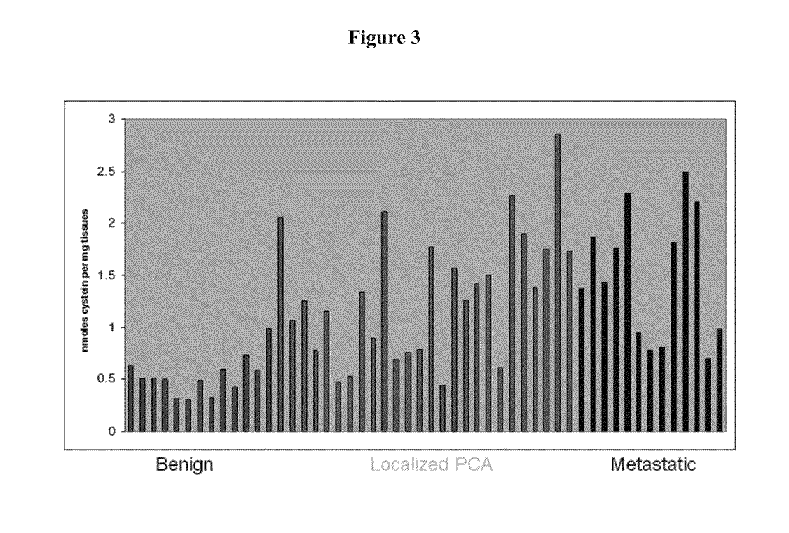

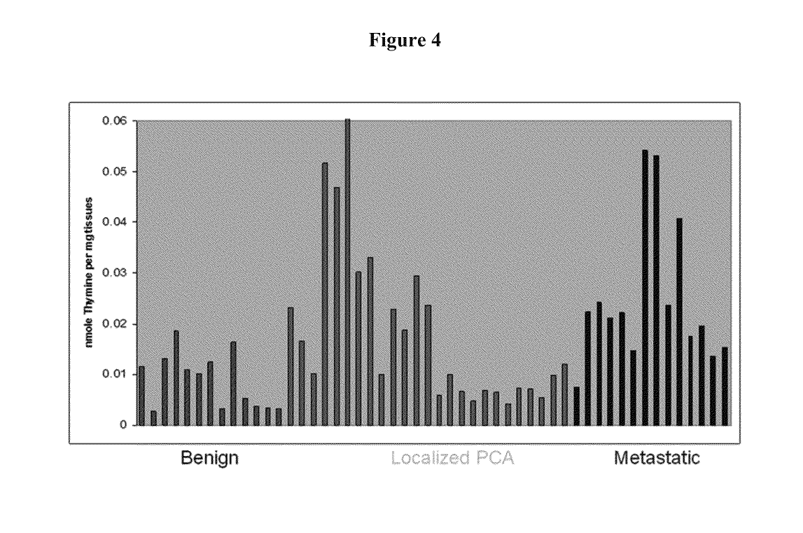

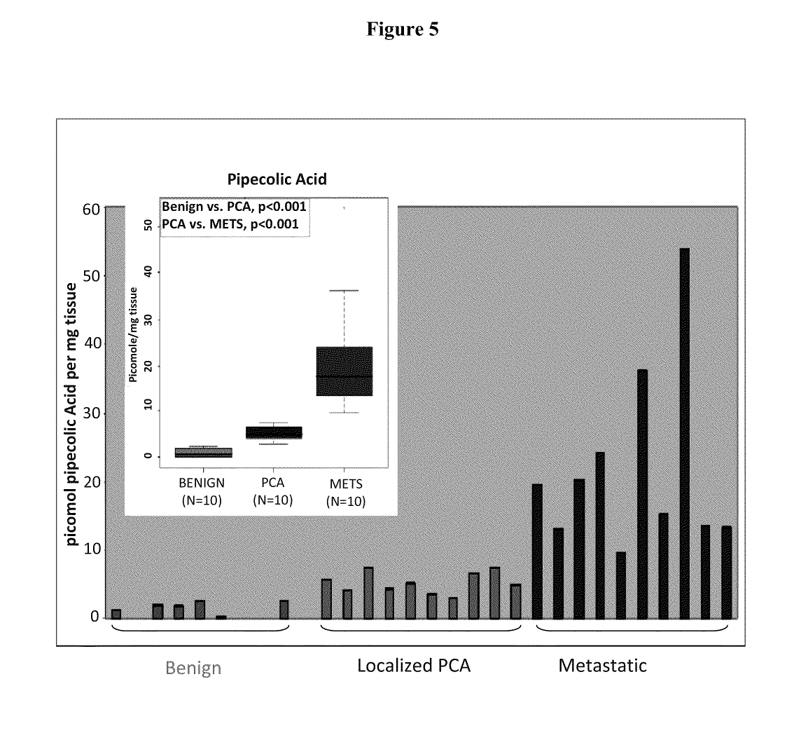

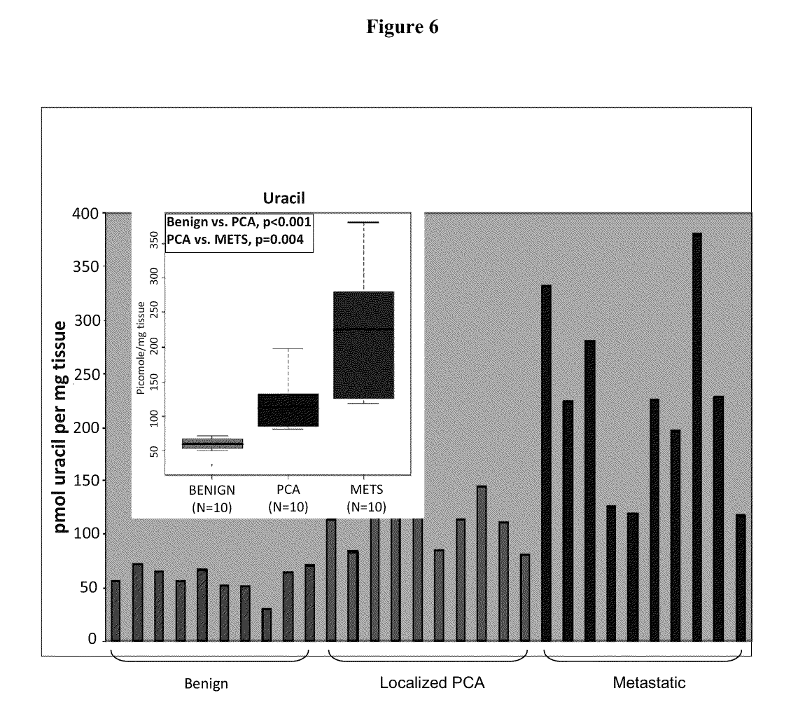

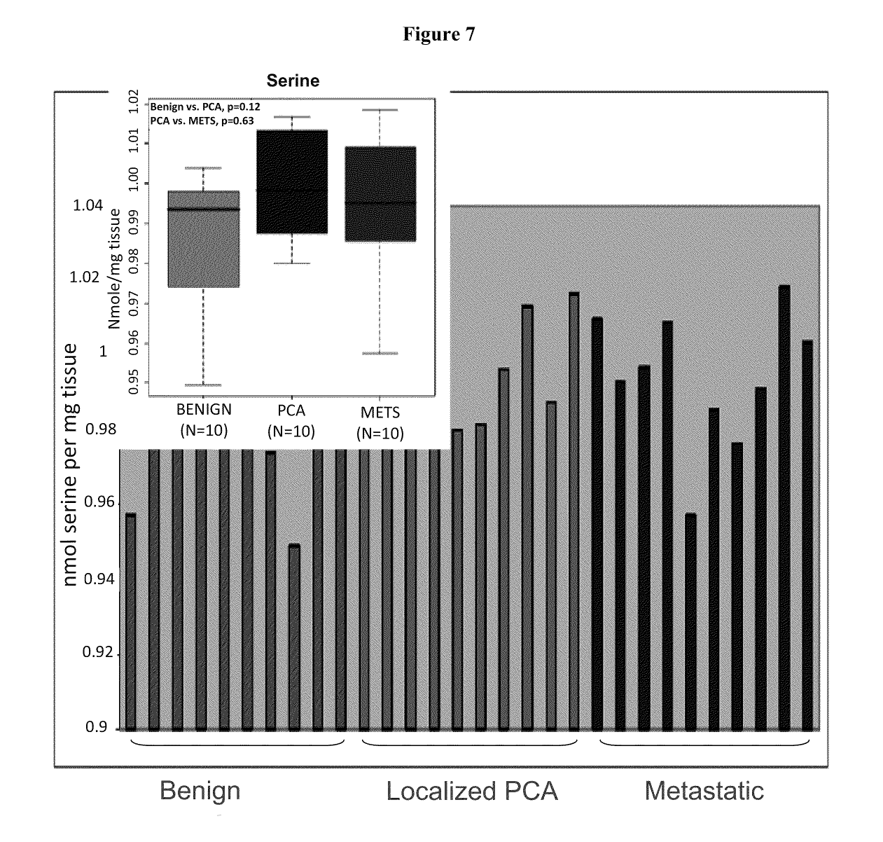

Localized prostate cancer-associated metabolites such as glutamic acid, glycine, cysteine, thymine, pipecolic acid, uracil and serine were quantified in prostate-derived tissue specimens. Using Stable Isotope Dilution (SID) Selected Ion Monitoring (SIM) GC-MS, we quantified target metabolites. First, the samples were modified to their t-butyl dimethylsilyl derivatives and analyzed with an Agilent 5975 MSD mass detector using Electron Impact (EI) ionization. Glutamic acid, cysteine, glycine and thymine were quantified in 52 prostate-derived samples. This included 13 benign adjacent (Benign), 26 localized prostate cancer (PCA), and 13 metastatic samples (Mets). For SIM analysis, the m/z for native and isotopically-labeled molecular peaks for various target metabolites quantified was: 406 and 407 (cysteine), 432 and 437 (glutamic acid), 218 and 219 (glycine), 297 and 301 (thymine), 198 and 207 (pipecolic acid) (n=30), 283 and 285 (uracil) (n=30) and 390 and 392 (serine) (n=30). The levels of metabolites were normalized to tissue weight. The levels of glutamic acid, glycine, cysteine, thymine, pipecolic acid and uracil are all elevated in localized PCA compared to benign prostate tissues (FIGS. 1-6). There is no change in the levels of serine, which do not vary during cancer progression (FIG. 7).

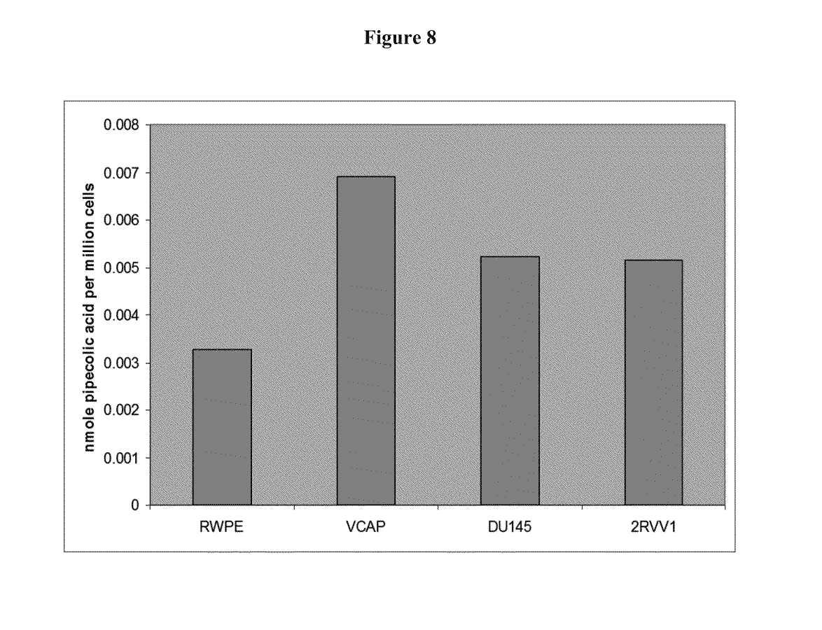

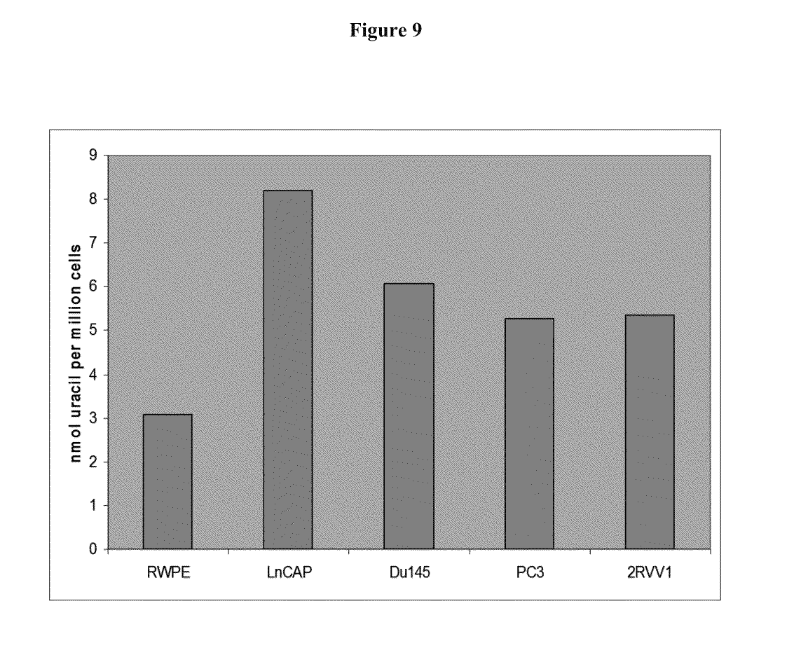

Prostate cancer cell lines were also used to validate the tissue data. Invasive prostate cancer cell lines (LnCaP, Du145, PC3 and 2RVV1) showed higher levels of pipecolic acid (FIG. 8) and uracil (FIG. 9) than non-invasive prostate cell line (RWPE).

### Validation of Multiple Metabolites in Urine:

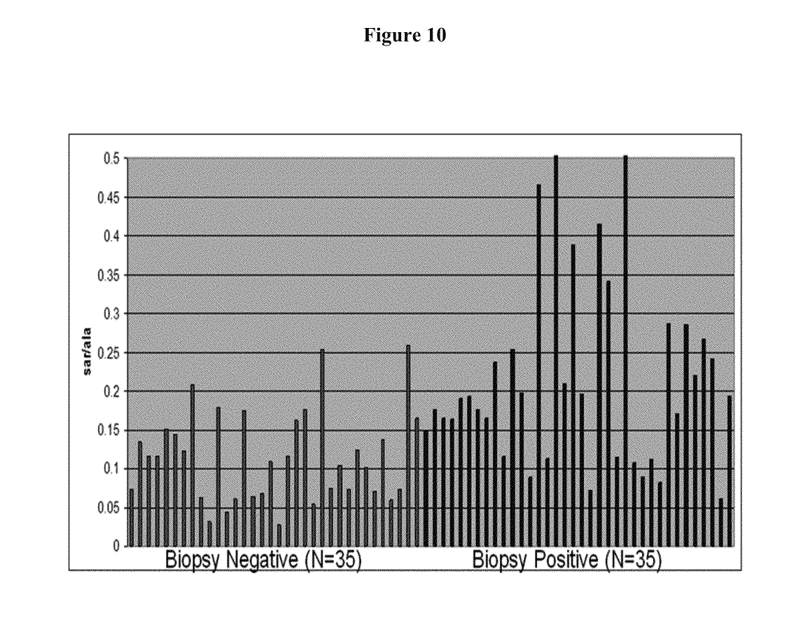

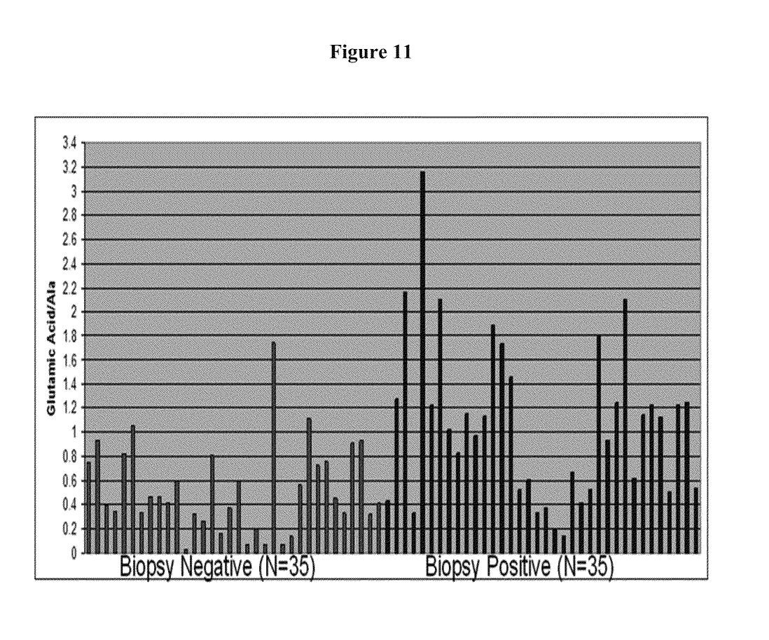

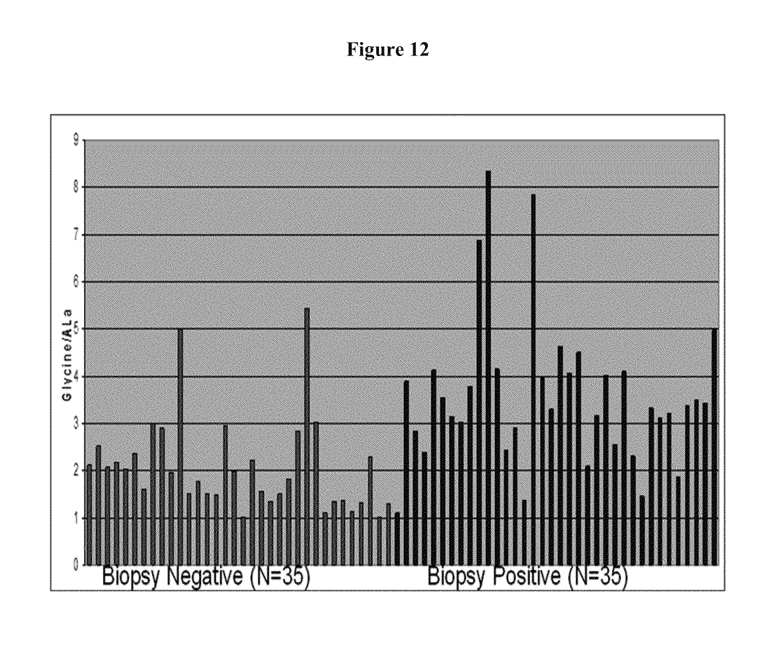

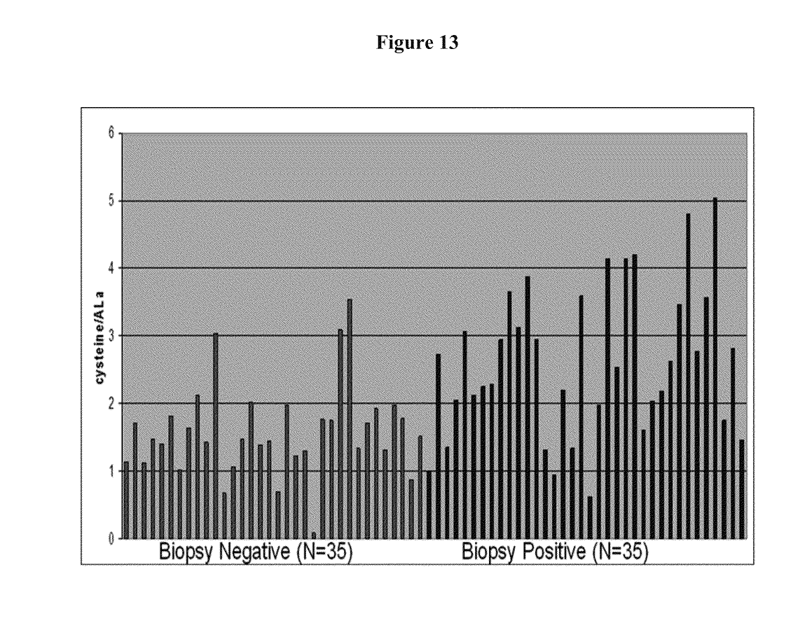

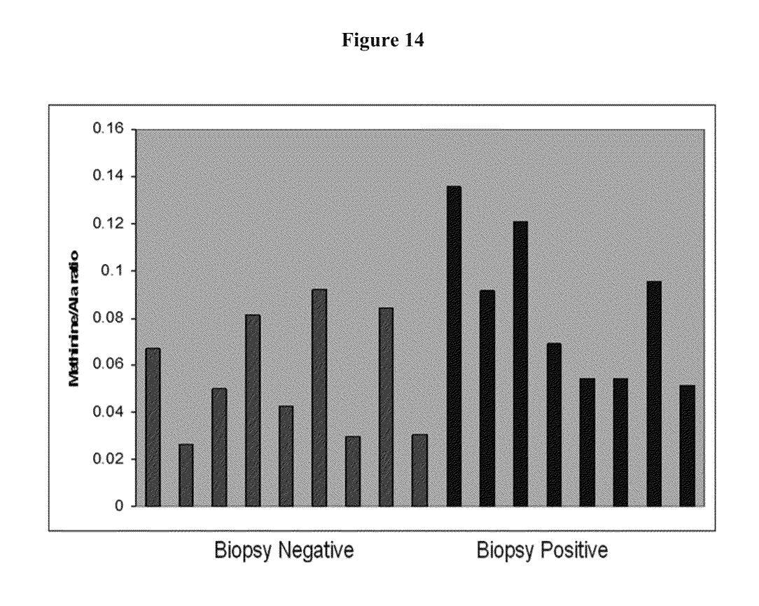

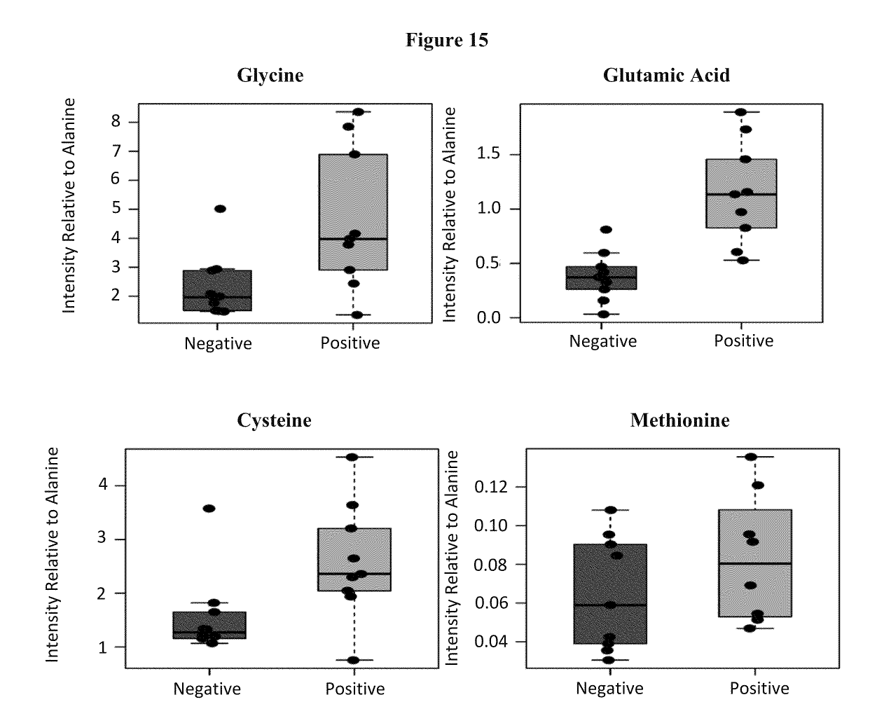

In order to investigate the potential of multiple metabolites as non-invasive prostate cancer markers, tissue-specific metabolites were used for validation in biopsy-positive and biopsy-negative urine sediments. A GC/MS methodology was developed to measure additional metabolites such as glutamic acid, glycine, cysteine and methionine. The levels of these metabolites were then analyzed in biopsy-positive and biopsy-negative urine sediments. Levels were reported as sarcosine/alanine, glutamic acid/alanine, glycine/alanine, cysteine/alanine, and methionine/alanine ratios. Alanine was used as internal control to normalize the levels of sarcosine, glutamic acid, glycine, cysteine and methionine in urine. The sarcosine/alanine ratio, glutamic acid/alanine ratio (Wilcoxon P=0.0003), glycine/alanine ratio (Wilcoxon P=0.0279, and cysteine/alanine ratio (Wilcoxon P=0.0133) of biopsy-positive urine sediments showed higher levels than corresponding biopsy-negative urine sediments (FIGS. 10-13). There was no change in the levels of methionine, which did not vary during cancer progression (FIG. 14). Box plots showed elevated levels of glutamic acid, glycine and cysteine in biopsy-positive urine sediments compared to the biopsy-negative controls (FIG. 15).

### Polyamines: Tissue Biomarkers for Aggressive Prostate Cancer

A simple and sensitive method for the simultaneous determination of polyamines (putrescine, spermidine, and spermine) in tissues, cell lines, and urine was developed. Polyamines were quantified by converting them into their N-heptafluorobutyl derivatives using GC-MS in Selected Ion-Monitoring (SIM) mode. The samples were modified to their heptafluorobutyl derivatives and analyzed with an Agilent 5975 MSD mass detector using electron impact ionization. Polyamines were initially quantified in 30 prostate-derived samples. This included 10 benign adjacent (Benign), 10 localized prostate cancer (PCA), and 10 metastatic samples (Mets).

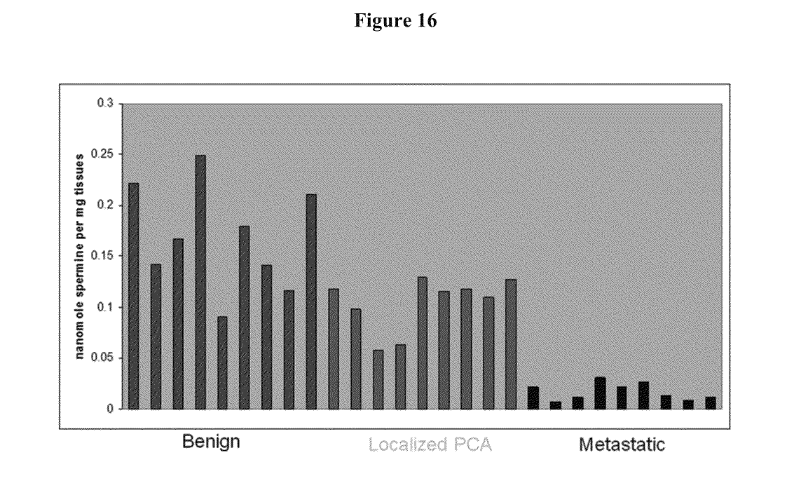

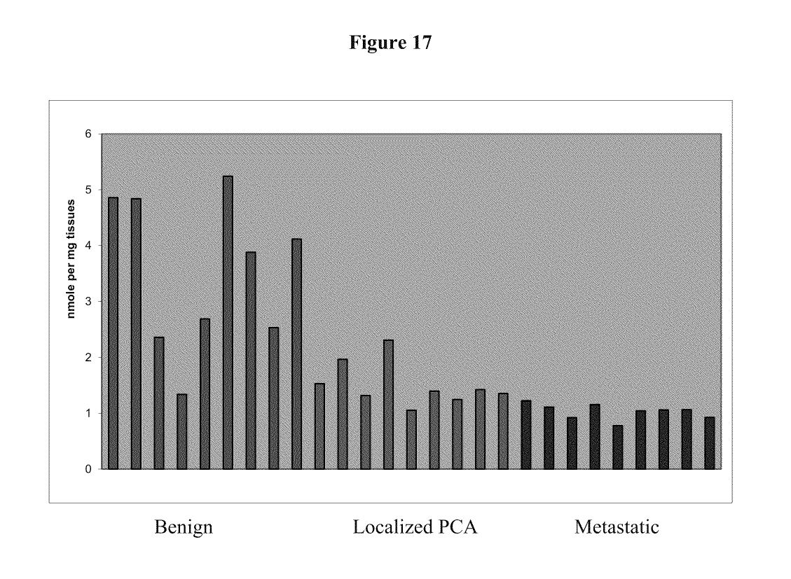

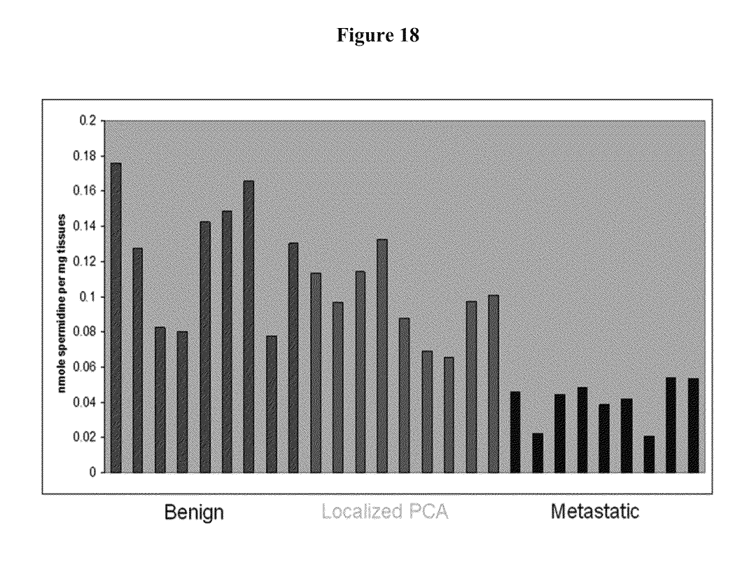

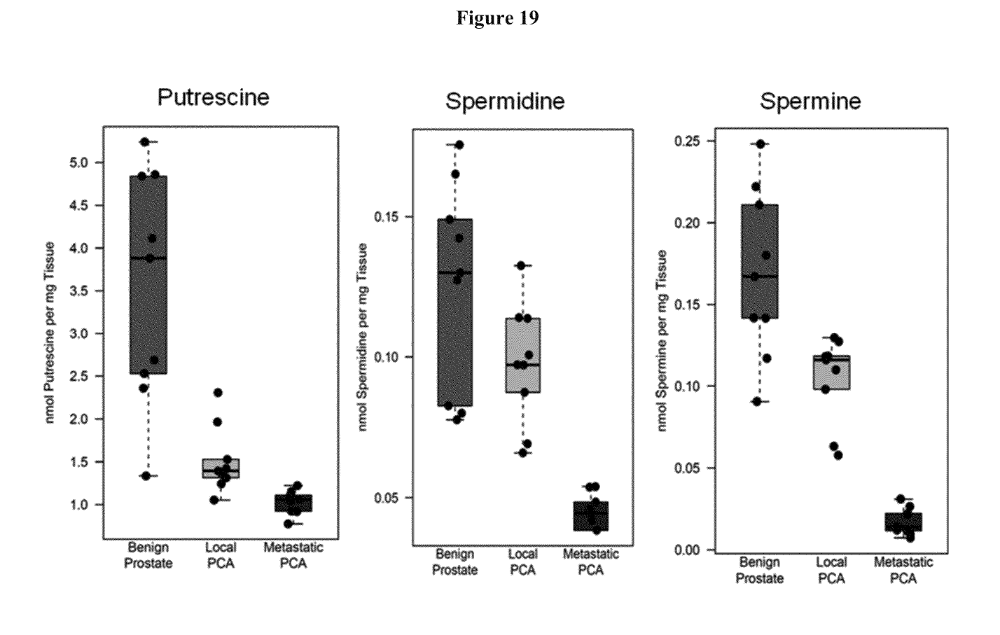

For SIM analysis, the m/z for native and labeled fragment ion peaks for various target metabolites were used for quantification. Values selected were 267 and 269 for putrescine, 323 and 331 for spermidine, and 576 and 584 for spermine. The levels were normalized by measuring the peak area of native and labeled ions. The levels of putrescine, spermidine, and spermine were normalized to tissue weight. The benign prostate tissues had elevated levels of spermine (PCA vs. Benign P=0.0018; PCA vs. Met, P=0.0059; Met vs. Benign, P=5.3×10⁻⁴), putrescine (PCA vs. Benign, P=0.0018; PCA vs. Met, 3.9×10⁻⁶ P=0.0059; Met vs. Benign, P=0.3×10⁻⁴), and spermidine (PCA vs. Benign, P=0.0731; PCA vs. Met P=1.3×10⁻⁵; Met vs. Benign P=8.5×10⁻⁵) compared to localized prostate cancer and metastatic prostate cancer tissues (FIG. 16-18). Box plots showed reduced levels of polyamines during cancer progression (FIG. 19). The P-values were calculated using a two-sided Welch two sample t-test to compare groups. The polyamine levels in tissues in prostate cancer are decreased during cancer progression, in contrast to many other cancer tissues (e.g., breast) in which polyamines metabolites increased with more aggressive cancer. Comparison of polyamine levels in benign and malignant tissues of human prostate showed that benign hyperplastic prostatic tissues have higher levels of spermine as compared to tumor tissue, especially in prostatic carcinoma with metastases. Hence, a dramatic decrease of the prostatic spermine content indicates a phenotypic conversion of prostatic tissue from a benign state to a malignant one. Therefore, experiments conducted during the course of development of some embodiments of the present invention show that polyamines find use as biomarkers for malignant behavior in prostate cancer. In some embodiments of the present invention, GC-MS-based polyamine validation constitutes a powerful, non-invasive method for the in vivo detection of polyamines in prostate cancer tissues.

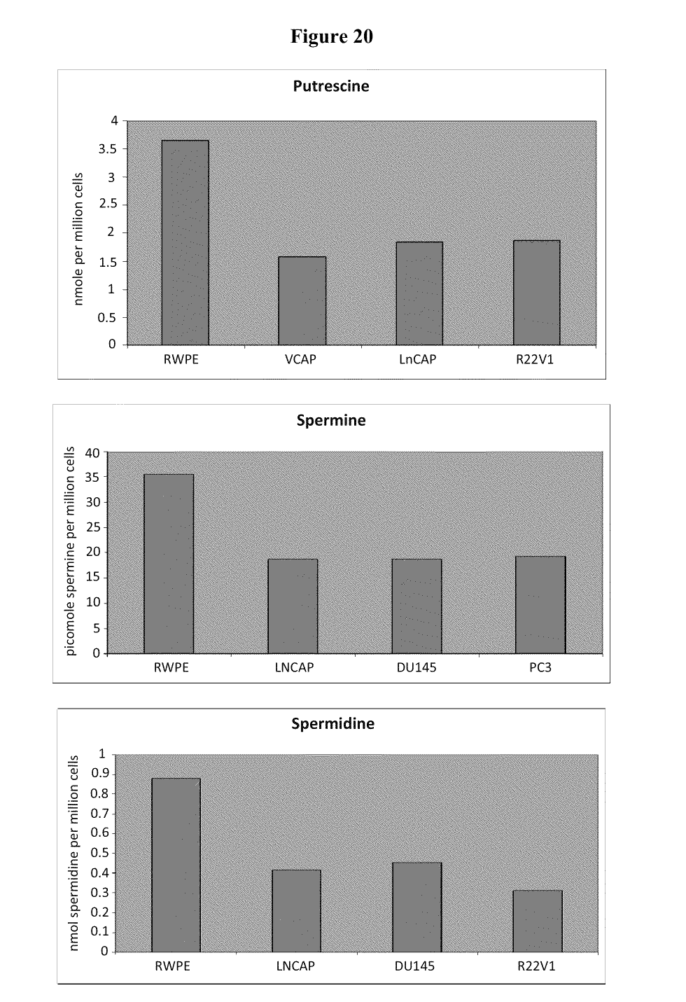

Cell line data validate observations made with tissue samples. Invasive (e.g., metastatic) prostate cancer cell lines (LNCaP, VCaP, DU145, PC3, and 2RVV1) showed lower polyamine levels than the normal prostate cell line (RWPE) (FIG. 20). Therefore, reduced levels of polyamines in aggressive prostate cancer demonstrate the utility of polyamines as prognostic markers.

### Polyamines: Noninvasive Metabolite Markers for Prostate Cancer in Urine

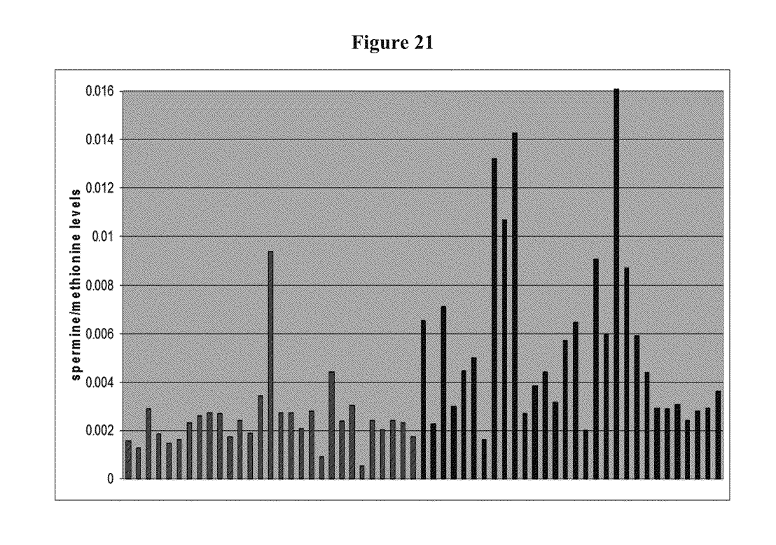

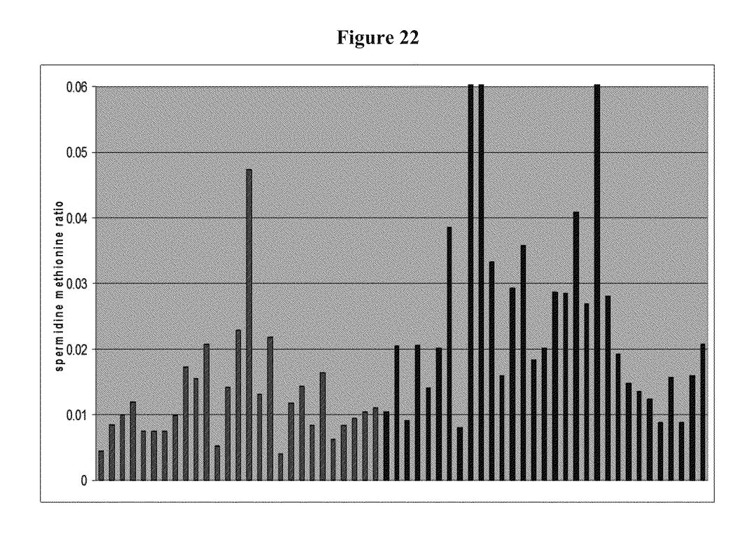

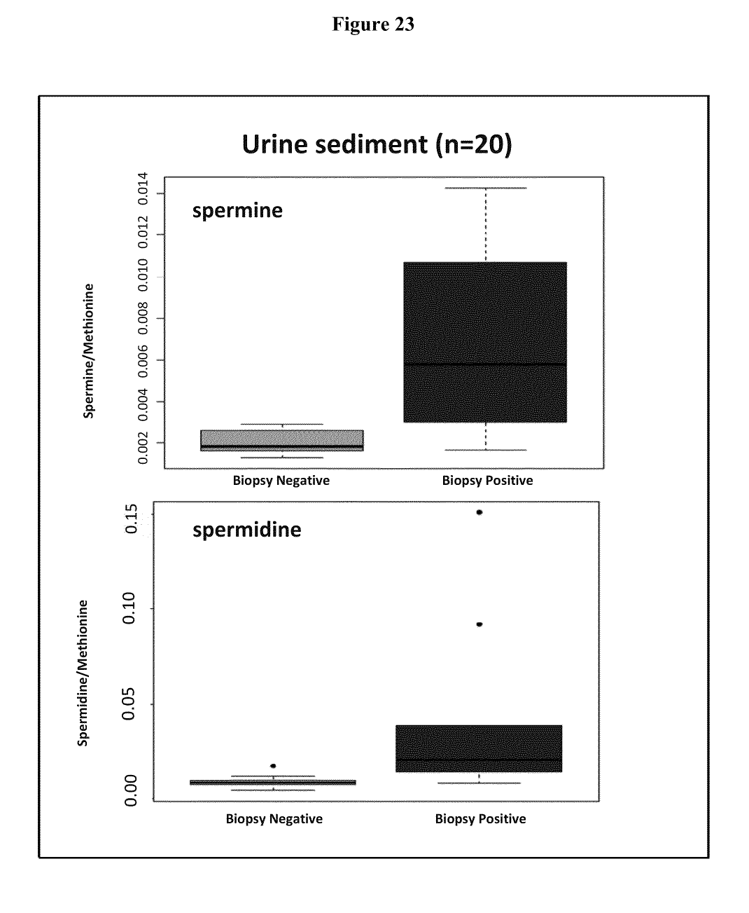

A GC-MS-based methodology was developed to quantify the levels of polyamines in tissues, as described supra. A modified GC/MS validation assay was also developed, and used to analyze polyamine levels in biopsy-positive and biopsy-negative urine sediments. Initially, the metabolites were converted to isobutyl esters by treating them with isobutanol, which were then modified to heptafluorobutyl esters. Both endogenous methionine and polyamines were derivatized and quantified. Methionine is used as a control to normalize polyamines. A GC-MS-based target metabolite assay was used to quantify the levels in urine sediments. Initially, 20 urine sediments (10 from each category: biopsy-positive and biopsy-negative) were used for quantification. The levels were reported as spermidine/methionine ratio and spermine/methionine ratio. The average spermine/methionine ratio (Wilcoxon P=0.003) and spermidine/methionine (Wilcoxon P=0.002) was significantly higher in the urine of biopsy-positive prostate cancer patients (n=10) as compared to the biopsy negative controls (n=10) (FIG. 21-22). Box plots showed elevated levels of spermine and spermidine in urine sediments from biopsy-positive individuals compared to those from biopsy-negative individuals (FIG. 23). While the present invention is not limited to any particular mechanism, and an understanding of the mechanism is not necessary to practice the present invention, it is contemplated that this is due to the secretion of polyamines from prostate tissues to urine during cancer progression. These results indicate that polyamines find use as non-invasive markers for detection of prostate cancer.

### Validation of Fatty Acids in Tissues:

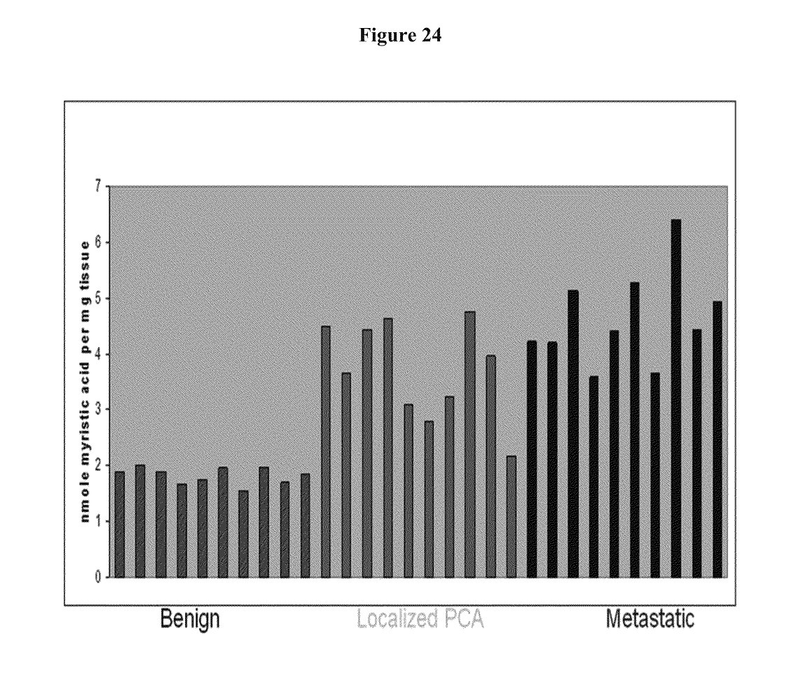

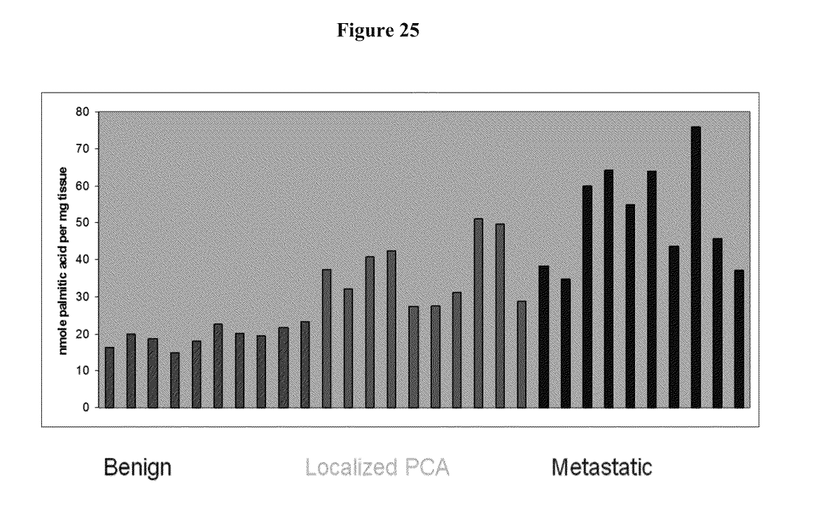

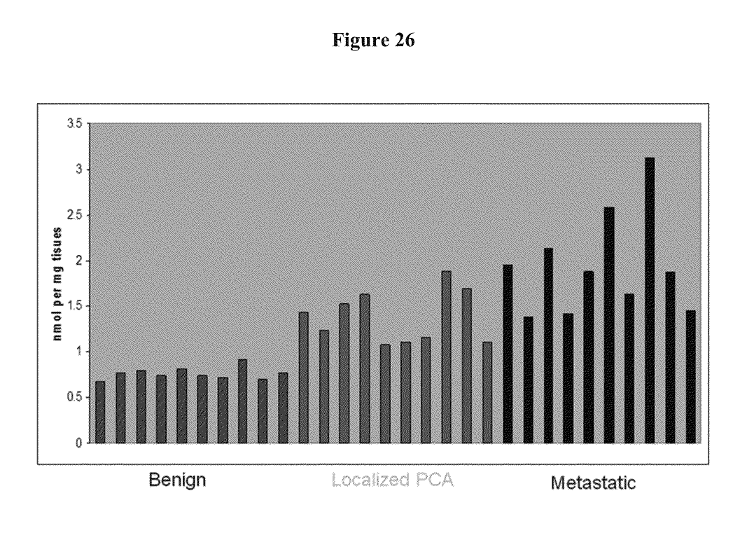

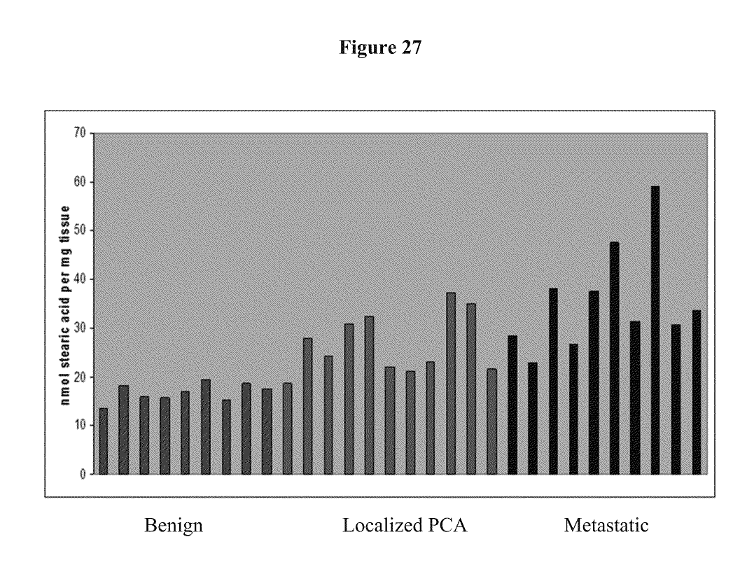

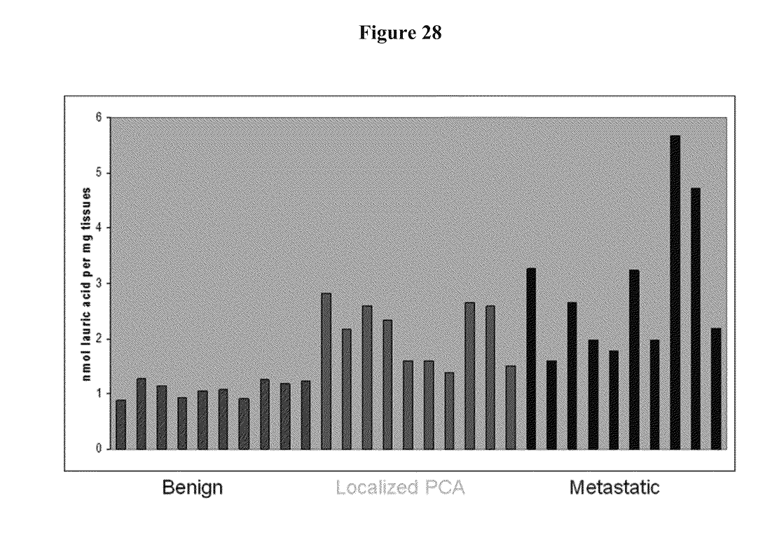

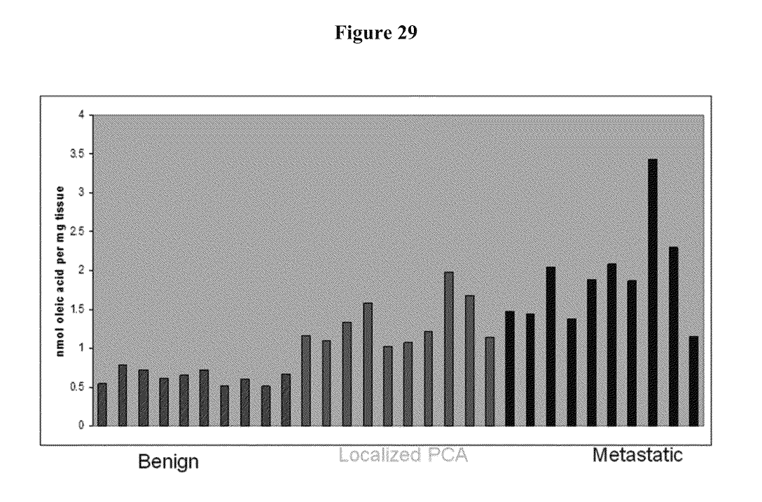

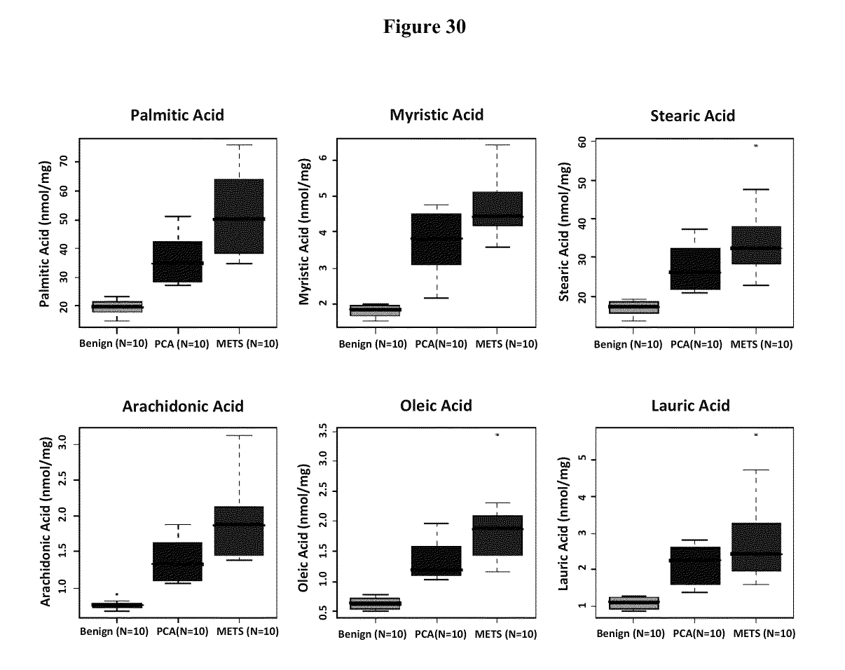

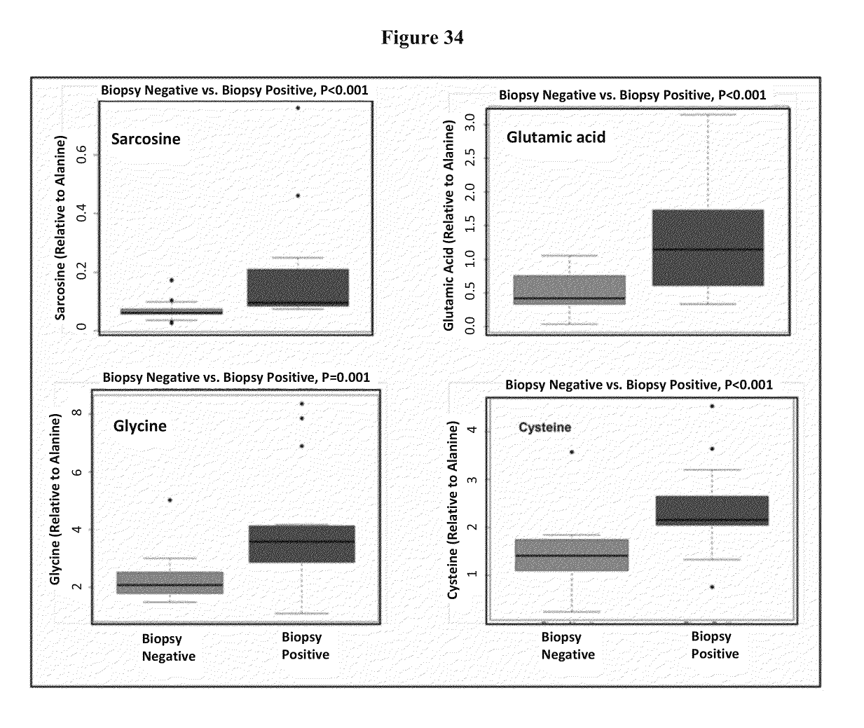

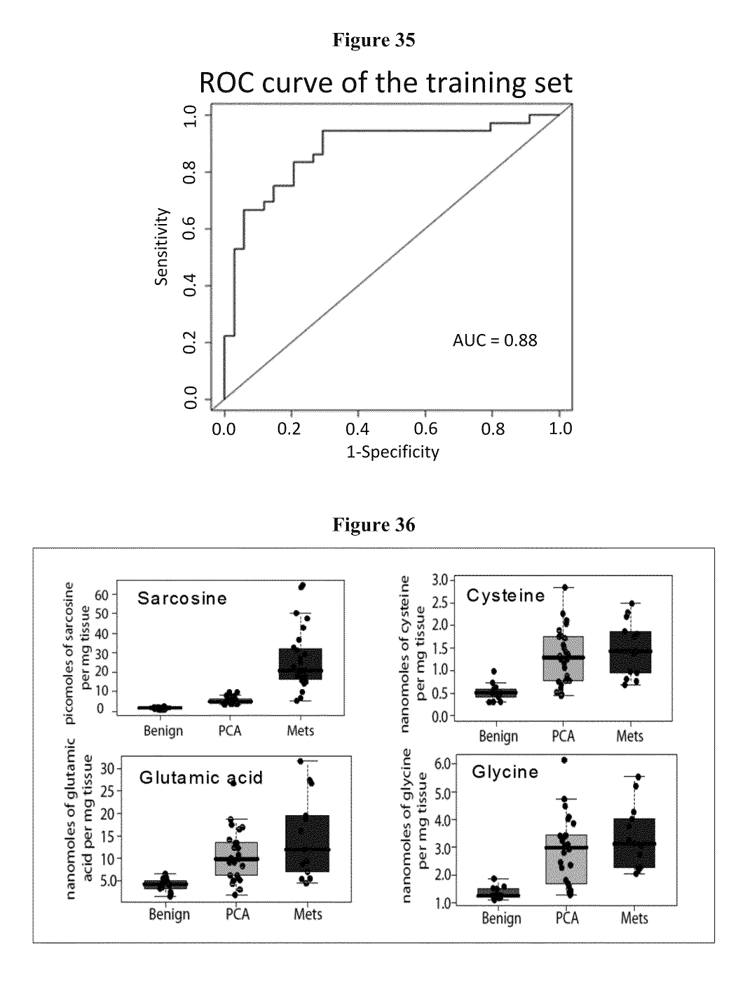

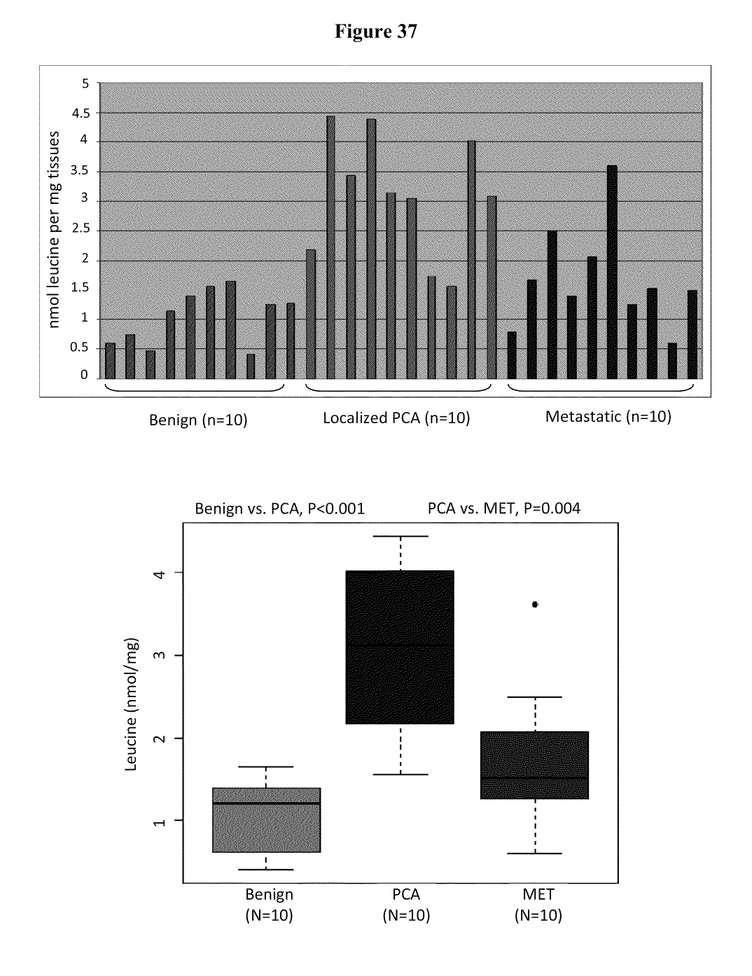

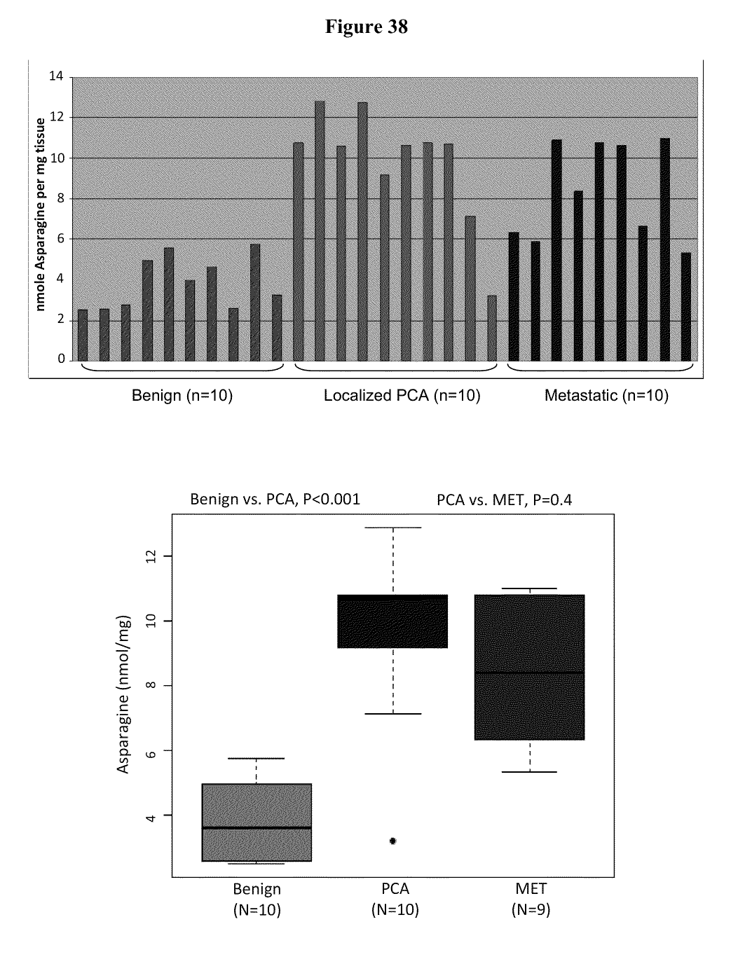

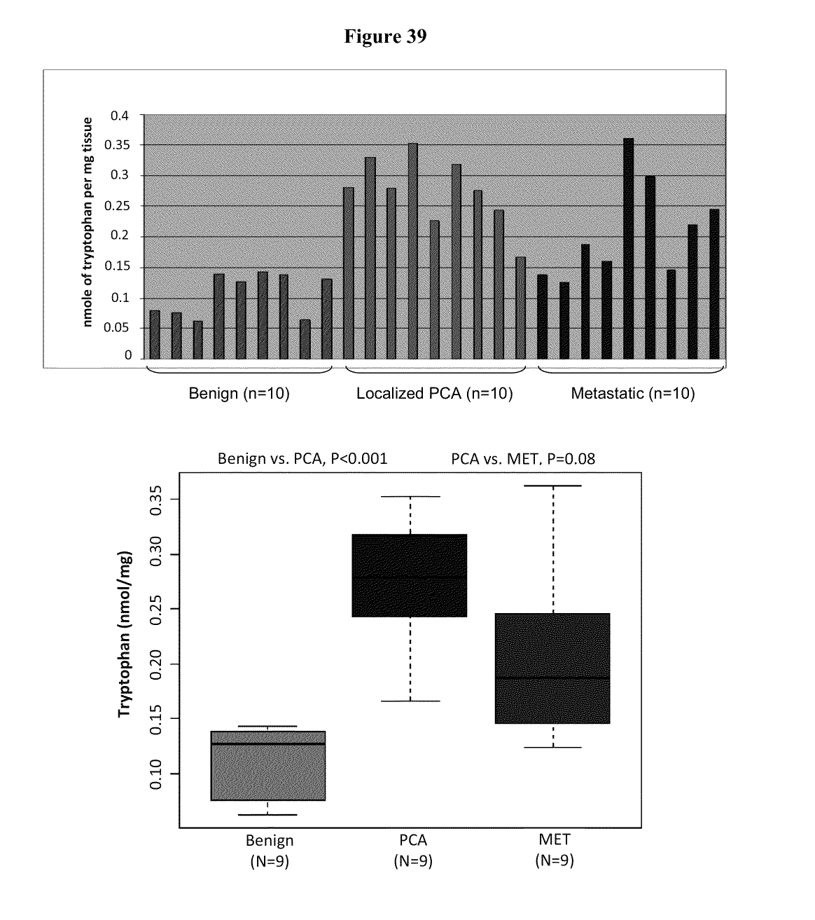

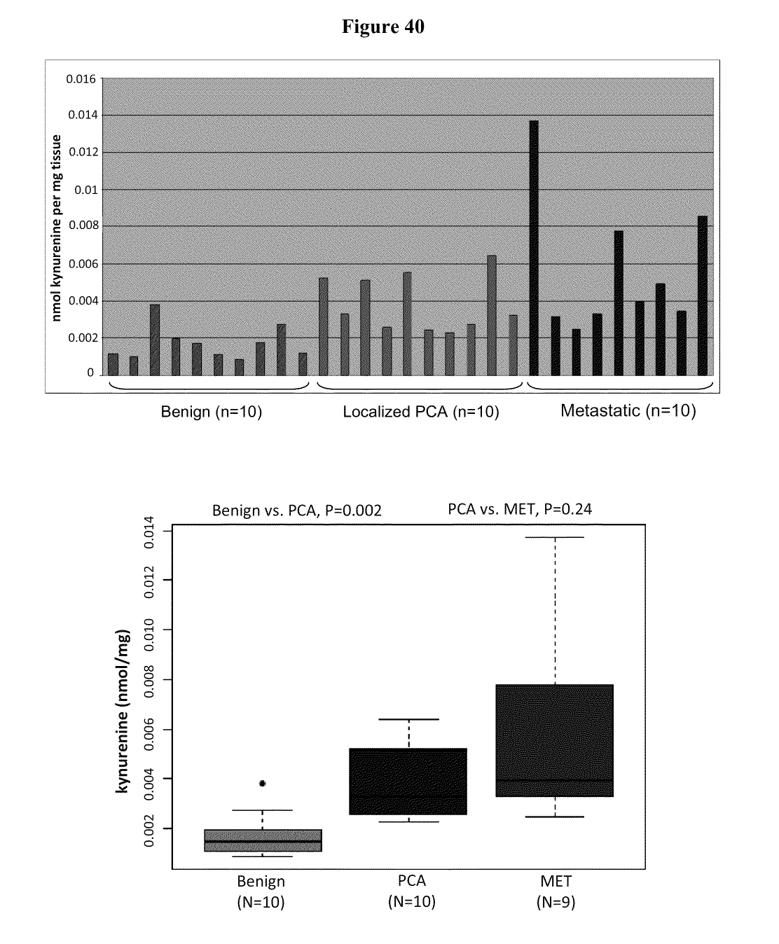

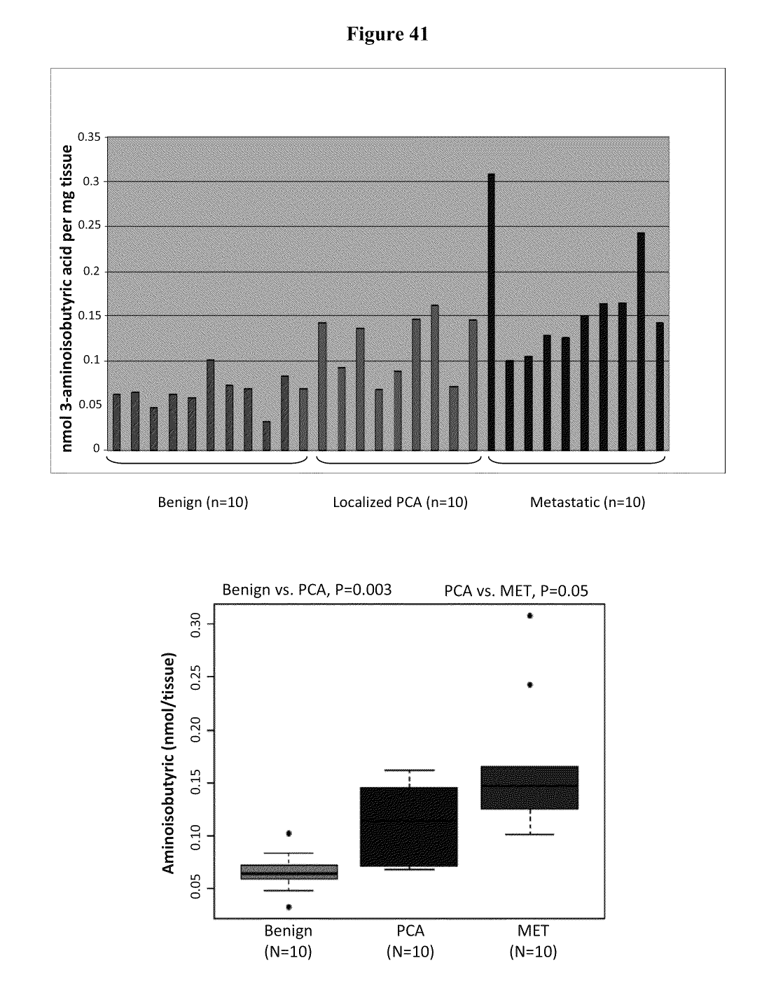

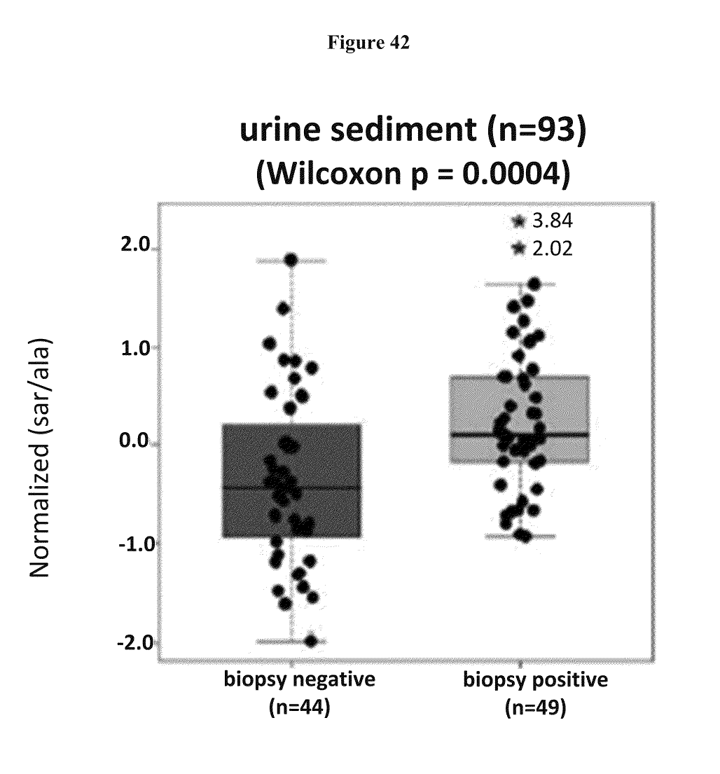

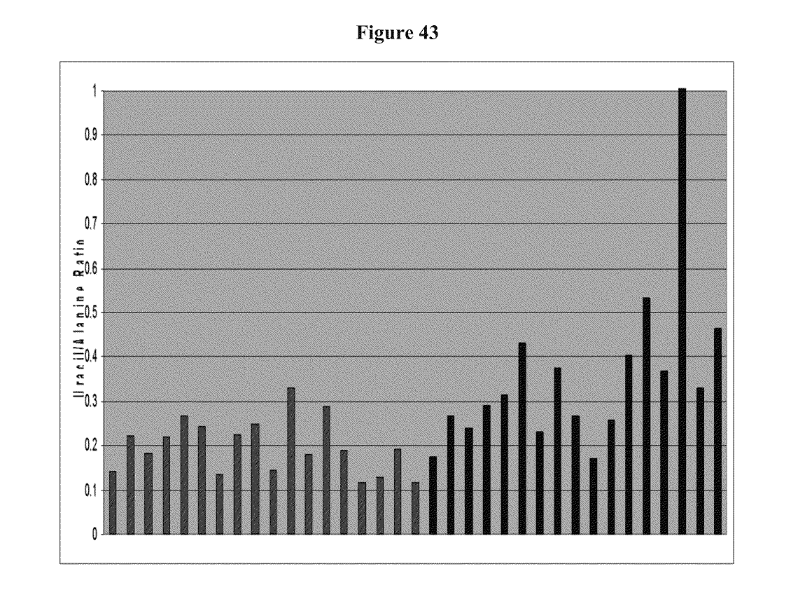

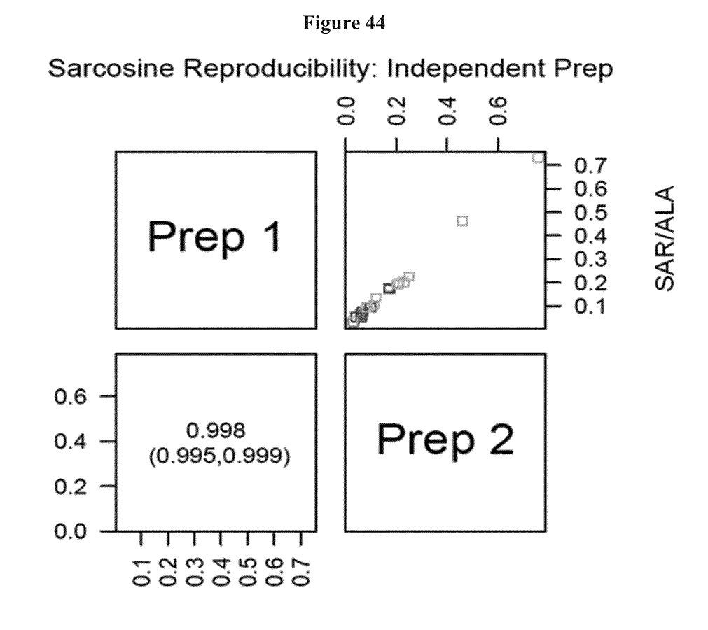

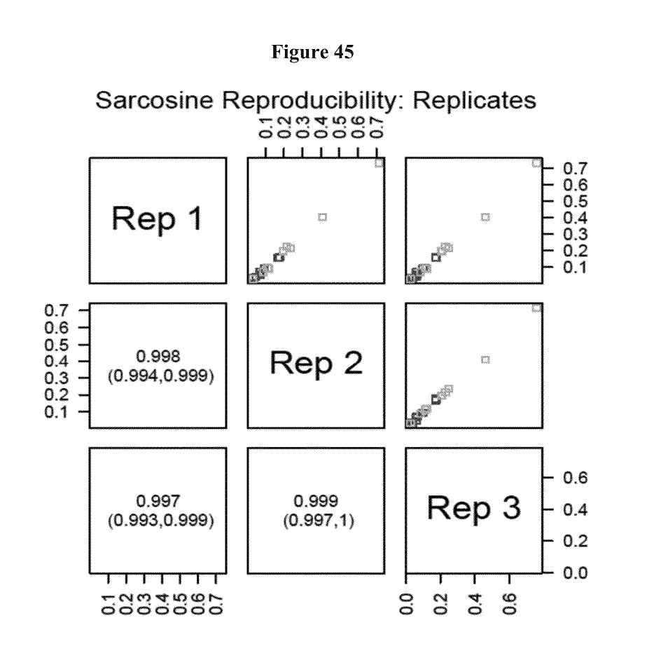

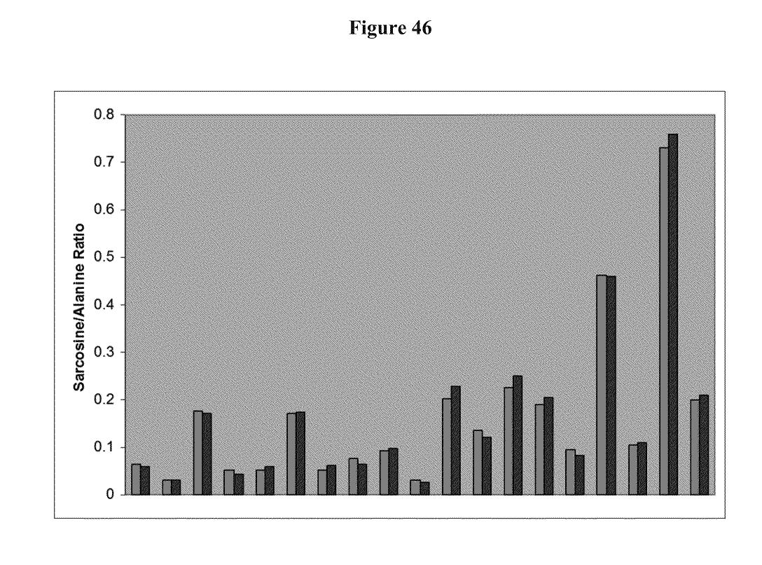

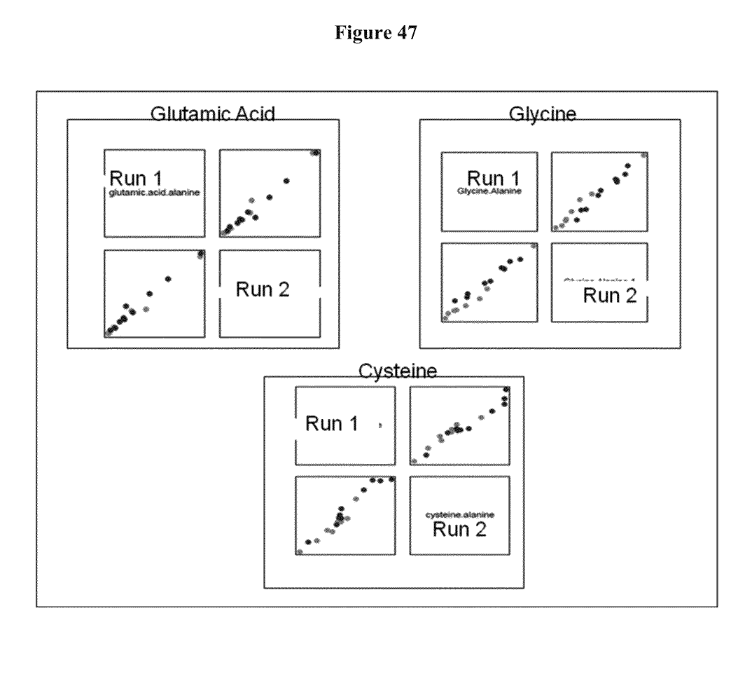

Fatty acids (myristic acid, stearic acid, palmitic acid, oleic acid, arachidonic acid and lauric acid) were also quantified in prostate-derived tissue specimens using Selected Ion Monitoring (SIM) GC-MS. First, the free fatty acids were modified to their t-butyl dimethylsilyl derivatives and analyzed using Electron Impact (EI) ionization with an Agilent 5975 MSD mass detector. Fatty acids were quantified in 30 prostate-derived samples (10 benign adjacent (Benign), 10 localized prostate cancer (PCA), and 10 metastatic samples (Mets). For SIM analysis, the m/z for native and isotopically-labeled molecular peaks for various target metabolites quantified was: 285 and 288 (myristic), 313 and 322 (palmitic acid), 341 and 359 (stearic), 339 and 341 (oleic), 369 and 372 (arachidonic), and 257 and 260 (lauric acid). The levels of metabolites were normalized to tissue weight. The levels of myristic acid, palmitic acid, arachidonic acid, stearic acid, lauric acid and oleic acid are all elevated during cancer progression (FIGS. 24-29). The box plots showed the elevated levels of fatty acids during prostate cancer progression (FIG. 30). FIGS. 34-47 show additional validation and characterization of markers and panels of markers useful in embodiments of the present invention.

Table 3 shows the AUC for individual markers and panels of markers of embodiments of the present invention.

TABLE 3

| Metabolites | AUC |
|---|---|
| Sarcosine | 0.76 |
| Glutamic Acid | 0.74 |
| Glycine | 0.79 |
| Cysteine | 0.73 |
| Multiplex Panel | 0.88 |

### Example 3

### Validation of Metabolites in Breast Cancer Tissues and Cell Lines

### 1. Sarcosine Validation in Breast Cancer Tissues:

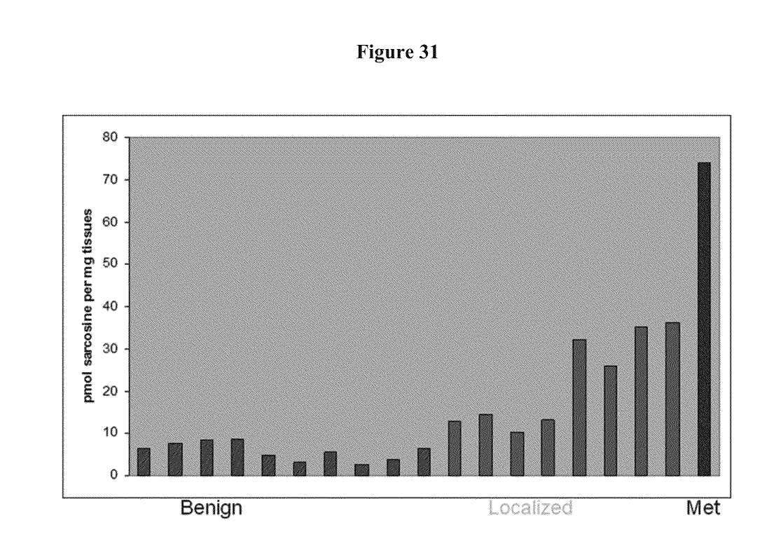

Sarcosine was identified as a differential metabolite that is highly elevated during prostate cancer progression. GC-MS studies indicate elevated levels of many metabolites upon cancer progression from benign to localized and subsequently metastatic disease. Analysis with cell lines supports this observation. The same strategy was applied to breast cancer samples. 19 tissue samples (10 benign, 8 localized and 1 metastatic breast cancer tissues) were analyzed. Breast cancer tissues showed higher sarcosine levels than the corresponding benign tissues (FIG. 31).

### 2. Validation of Sarcosine in an Invasive and Non-Invasive Breast Cell Lines:

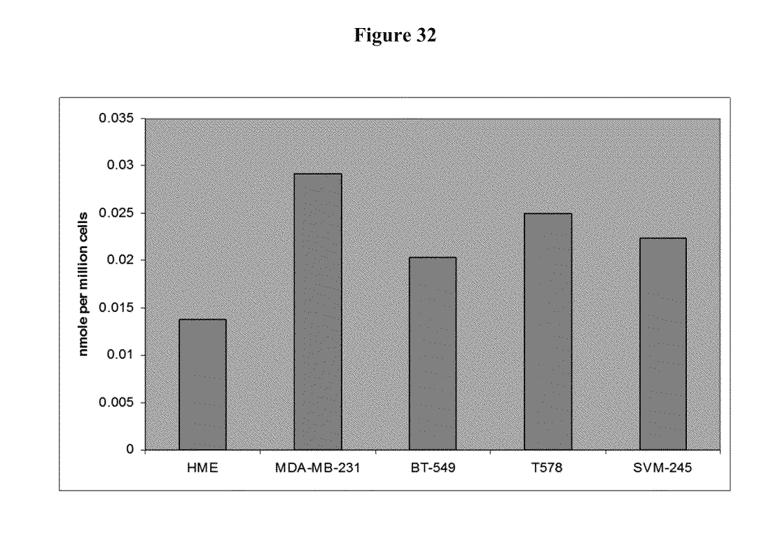

A series of Invasive (MDA-MB-231, BT-549, T578, SVM-245) and non invasive (HME) breast cell lines were used for sarcosine validation. The invasive cell lines exhibited higher sarcosine levels than the corresponding non-invasive cell line (FIG. 32).

### 3. Validation of Polyamines in Breast Cell Lines:

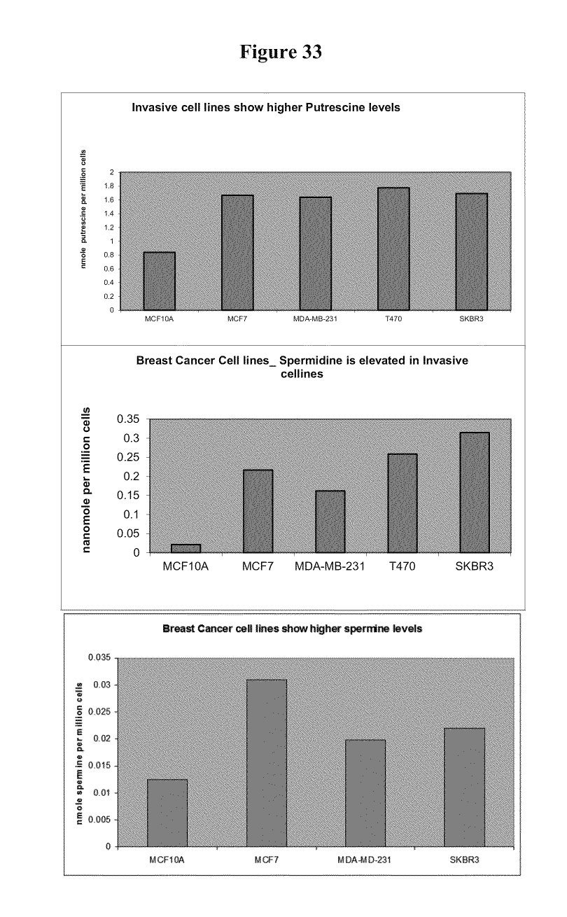

A GC-MS methodology was developed to validate polyamines (putrescine, spermidine and spermine) in a set of breast cancer cell lines. Invasive cell lines (MCF7, MDA-MB-231, T470, SKBR3) showed elevated levels of putrescine, spermidine and spermine in comparison to a corresponding normal cell line (MCF 10A) (FIG. 33).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of diagnosing prostate cancer, comprising: a) detecting the level of sarcosine, glutamic acid, glycine and cysteine in a urine sample from a subject; and b) diagnosing prostate cancer when the levels of sarcosine, glutamic acid, glycine and cysteine are elevated relative to the level in a non-cancerous subject.

2. The method of claim 1, wherein said method further comprises the step of detecting the level of one or more metabolites selected from the group consisting of acetyl glucosamine, kyurenine, uracil, homocysteine, asparagine, glutamic acid, sperminide, spermine, 2-aminoadipic acid, leucine, proline, threonine, maleate, histidine, citrulline, adenosine and inosine.

3.-10. (canceled)

11. A method of diagnosing prostate cancer, comprising: a) detecting the presence or absence of one or more cancer specific metabolites selected from the group consisting of pipecolic acid, serine, a polyamine, and a fatty acid in a urine sample from a subject; and b) diagnosing prostate cancer based on the presence or absence of said cancer specific metabolite in said urine sample.

12. The method of claim 11, wherein said polyamine is selected from the group consisting of putrescine, spermidine, and spermine.

13. The method of claim 11, wherein said fatty acid is selected from the group consisting of myristic acid, palmitic acid, arachidonic acid, stearic acid, lauric acid, and oleic acid.

14. The method of claim 11, wherein said urine sample is a urine sediment sample.

15. The method of claim 11, wherein said one or more cancer specific metabolites is present in cancerous samples but not non-cancerous samples.

16. The method of claim 11, wherein said one or more cancer specific metabolites is absent in cancerous samples but present in non-cancerous samples.

17. The method of claim 11, comprising the simultaneous detection of the presence or absence of more than one said cancer specific metabolites.
